(19) United States
(12) Patent Application Publication
(10) Pub. No.: US 2026/0048081 A1
(43) Pub. Date: Feb. 19, 2026

(54) COMBINED TREATMENT COMPOSITION FOR TUMOR TREATMENT AND COMBINED TREATMENT METHOD

(71) Applicants: ADVACCINE (SUZHOU) BIOPHARMACEUTICALS CO., LTD., Suzhou (CN); FUDAN UNIVERSITY, Shanghai (CN)

(72) Inventors: Bin Wang, Suzhou (CN); Gan Zhao, Suzhou (CN); Shuting Wu, Suzhou (CN); Lunan Zhang, Suzhou (CN); Zhihua Kou, Suzhou (CN)

(21) Appl. No.: 19/104,115

(22) PCT Filed: Aug. 15, 2023
(86) PCT No.: PCT/CN2023/113027

§ 371 Date: Feb. 14, 2025

(30) Foreign Application Priority Data
Aug. 17, 2022 (CN) 202210985077.3

(51) Int. Cl. A61K 33/243; A61K 9/00; A61K 31/4745; A61K 31/555; A61K 31/7088; A61K 45/06; A61P 35/00

(52) U.S. Cl. CPC A61K 33/243; A61K 9/0019; A61K 31/4745; A61K 31/555; A61K 31/7088; A61K 45/06; A61P 35/00

(57) ABSTRACT

The present invention relates to a combined treatment composition for tumor treatment and a combined treatment method. The combined treatment composition or a medicine kit comprises a therapeutically effective amount of a platinum chemotherapeutic drug, and a therapeutically effective amount of CpG oligonucleotide and a therapeutically effective amount of R848. The present invention further provides a combined treatment method for treating a tumor by means of the combined treatment composition or the medicine kit. Firstly, a low-dose chemotherapeutic drug is used to kill a tumor so as to release a tumor neoantigen, such that a body immune system autonomously identifies and screens the neoantigen, and then CpG oligonucleotide and an R848 activator of immune response are used to induce and promote the body to generate immune response specific to the tumor neoantigen, such that the anti-tumor function of T cells is improved and enhanced, thereby achieving the effect of inhibiting tumor growth.

### TECHNICAL FIELD

The present invention belongs to the technical field of medicines, and particularly relates to a combination therapy composition and a combination therapy method for treating a tumor.

### BACKGROUND

Cancer is still one of the major diseases threatening human health in the society today. According to the estimated data from the International Agency for Research on Cancer (IARC) of the World Health Organization, there were 19.29 million new cases of cancer worldwide in 2020, and the cancer deaths reached up to 9.96 million.

The combination of targeted therapy with chemotherapy and/or radiotherapy has become an important treatment means for tumors. The side effects of chemotherapeutic drugs are great, and patients often give up the treatment because they cannot tolerate the side effects of chemotherapy; although the targeted therapy has achieved a good curative effect on a plurality of tumors, it cannot be suitable for all tumors because mutations in key genes of the tumors can cause the treatment to be ineffective. Tumor immunotherapy has been an important research direction for tumor therapy in recent years, but immune tolerance (or immune neglect) is one of the root causes of the survival and uncontrolled growth of tumor cells in human tissues, and how to enhance the immunogenicity of anti-tumor cells so that the immune system can no longer “neglect” the tumor cells has been a difficult and key problem to overcome cancer.

The increase in neoantigens generated by tumor mutations is closely related to the formation of tumor-associated antigens (TAAs) and tumor-specific antigens (TSAs), and the neoantigens have higher immunogenicity and can increase T cell infiltration, thereby generating anti-tumor effects. However, immunotherapy regimens based on neoantigens, TAAs, and TSAs have a significant drawback, that is, the therapeutic effect is unstable. The reason for this is that the screened and designed antigens cannot ensure that they can fully exert effective anti-tumor immune responses and effects, and further cannot keep up with the pace of tumor antigen mutations. The successful application of PD-1 antibodies in tumor therapy shows that the internal immune system of an organism is stimulated to have an anti-tumor effect, and a better anti-tumor effect can be achieved only by improving and awakening the function of these T cells. Vaccination in situ is a promising approach to cancer immunotherapy, which involves blocking the mechanisms by which cancer cells escape detection by the immune system, independent of specific tumor-associated antigens. This approach may elicit a broad immune response to the cancer cells, resulting in long-term remission or cure. However, one of the important limiting factors is that the tumor antigen induced in situ is not sufficient to induce a strong anti-tumor immune response and reaction, so it is often seen in the art that the anti-tumor effect is not significant and consistent. This technology requires a great improvement in the antigen-releasing ability and the activation of an anti-tumor immune response.

The use of chemotherapeutic drugs can cause cellular DNA damage, kill some tumor cells, contribute to increasing a mutation burden, and induce the generation of subclonal neoantigens. The neoantigens only provide immunogenicity for anti-tumor immunity and cannot effectively induce the body to generate the anti-tumor immunity, and appropriate molecules are needed to activate the immune system of the body.

### SUMMARY

Based on the above, in order to further promote the therapeutic effect on tumors, the present invention uses chemotherapeutic drugs to induce the generation of neoantigens, enables the immune system of a tumor patient to autonomously identify and screen the neoantigens, and simultaneously promotes and strengthens the anti-tumor function of T cells, thereby achieving a final treatment regimen with a good anti-tumor effect. The novel therapy of the present invention can fully utilize the tumor antigen release effect in the killing process caused by chemotherapy, and by using the combination therapy of Toll-like receptor (TLR) activators, provides a new idea for tumor treatment.

The TLR activators are used for treating tumors and are currently mostly used as adjuvants in personalized tumor vaccines. Some are used in combination with conventional chemotherapeutic drugs or with immune checkpoint-targeted drugs, thereby remarkably improving the therapeutic effect on tumors. However, there is no related pharmaceutical research on improving tumor-specific cellular immune responses by using “low-dose chemotherapeutic drugs+TLR activators” at present. Unlike other existing immunotherapeutic strategies, the therapeutic concept of the present invention is unique in that: it can be free from the limitation of tumor types and tumor antigen mutations, the tumor antigens are released by small-dose chemotherapy, and the immune activators induce tumor individuals to generate specific tumor immunity, thereby improving the therapeutic effect on tumors.

The present invention innovatively utilizes the immunological activation mechanism of TLR activators, enables the immune system of a tumor patient to autonomously identify and screen the neoantigens, simultaneously promotes and strengthens the anti-tumor function of T cells, and inhibits the interference of the tumor immune microenvironment, thereby achieving a final treatment regimen with a good anti-tumor effect. In order to further promote the therapeutic effect on tumors, the tumor antigen release effect in the killing process caused by chemotherapeutic drugs is fully utilized, and the combination therapy of activating the internal anti-tumor immunity in a body by TLR activators is also used, so that the therapeutic effect on the tumor is improved. The combination therapy of the present invention has an innovative theory and good clinical application prospects.

In view of the above, the present invention is proposed.

The present invention aims to provide a pharmaceutical composition for tumor combination therapy and a combination therapy method for treating a tumor by the pharmaceutical composition for combination therapy, which is expected to realize a broad-spectrum anti-tumor effect.

In order to solve the above technical problems and achieve the above purpose, the present invention provides the following technical solutions:

In one aspect, the present invention provides a pharmaceutical composition for tumor combination therapy, comprising a therapeutically effective amount of a platinum-based chemotherapeutic drug, a therapeutically effective amount of a CpG oligonucleotide, and a therapeutically effective amount of R848.

In an alternative embodiment, the platinum is selected from one or more of cisplatin, carboplatin, nedaplatin, oxaliplatin, and lobaplatin. Preferably, the platinum is cisplatin.

In an alternative embodiment, the therapeutically effective amount of the CpG oligonucleotide (CpG ODN) is a B-class CpG ODN or a C-class CpG ODN.

Preferably, the B-class CpG ODN is selected from one or more of ODN 1826, ODN 1018, and ODN 2006/7909.

Preferably, the C-class CpG ODN is selected from one or more of ODN M362, ODN 2395, and D-SL03.

In an alternative embodiment, the therapeutically effective amount of R848 is selected from one or more of water-soluble R848 and lipid-soluble R848.

In an alternative embodiment, the therapeutically effective amount of the platinum-based chemotherapeutic drug is administered at a dose of 2 μg/kg to 4000 μg/kg.

In an alternative embodiment, the therapeutically effective amount of the CpG oligonucleotide is administered at a dose of 0.001 mg/time to 32 mg/time.

In an alternative embodiment, the therapeutically effective amount of R848 is administered at a dose of 0.001 mg/time to 5 mg/time.

In an alternative embodiment, the therapeutically effective amount of the platinum-based chemotherapeutic drug is administered at a dose of 2 μg/kg to 20 μg/kg;
the therapeutically effective amount of the CpG oligonucleotide is administered at a dose of 0.001 mg/time to 32 mg/time;
the therapeutically effective amount of R848 is administered at a dose of 0.001 mg/time to 1 mg/time.

In a preferred embodiment, the therapeutically effective amount of the platinum-based chemotherapeutic drug is administered at a dose of 2 μg/kg to 400 μg/kg;
the therapeutically effective amount of the CpG oligonucleotide is administered at a dose of 0.05 mg/time to 2 mg/time;
the therapeutically effective amount of R848 is administered at a dose of 0.01 mg/time to 1 mg/time.

In another aspect, the present invention also provides a treatment method for treating a subject suffering from a tumor or cancer by the above pharmaceutical composition, the method comprising administering to the subject a therapeutically effective amount of the pharmaceutical composition; the pharmaceutical composition comprises a therapeutically effective amount of a platinum-based chemotherapeutic drug, a therapeutically effective amount of a CpG oligonucleotide, and a therapeutically effective amount of R848.

In an alternative embodiment, the platinum is selected from one or more of cisplatin, carboplatin, nedaplatin, oxaliplatin, and lobaplatin. Preferably, the platinum is cisplatin.

In an alternative embodiment, the therapeutically effective amount of the CpG oligonucleotide (CpG ODN) is a B-class CpG ODN or a C-class CpG ODN.

Preferably, the B-class CpG ODN is selected from one or more of ODN 1826, ODN 1018, and ODN 2006/7909.

Preferably, the C-class CpG ODN is selected from one or more of ODN M362, ODN 2395, and D-SL03.

In an alternative embodiment, the therapeutically effective amount of R848 is selected from one or more of water-soluble R848 and lipid-soluble R848.

In an alternative embodiment, the therapeutically effective amount of the platinum-based chemotherapeutic drug is administered at a dose of 2 μg/kg to 4000 μg/kg.

In an alternative embodiment, the therapeutically effective amount of the CpG oligonucleotide is administered at a dose of 0.001 mg/time to 32 mg/time.

In an alternative embodiment, the therapeutically effective amount of R848 is administered at a dose of 0.001 mg/time to 5 mg/time.

In an alternative embodiment, the therapeutically effective amount of the platinum-based chemotherapeutic drug, the therapeutically effective amount of the CpG oligonucleotide, and the therapeutically effective amount of R848 are sequentially administered to a subject in need thereof.

In an alternative embodiment, the administration of the therapeutically effective amount of the platinum-based chemotherapeutic drug is performed prior to the administrations of the therapeutically effective amount of the CpG oligonucleotide and the therapeutically effective amount of R848.

In an alternative embodiment, the administrations of the therapeutically effective amount of CpG oligonucleotide and the therapeutically effective amount of R848 are performed simultaneously.

In an alternative embodiment, the therapeutically effective amount of the platinum-based chemotherapeutic drug is administered 1 time, and the therapeutically effective amount of the CpG oligonucleotide and the therapeutically effective amount of R848 are administered 1-4 times for 1 administration cycle.

In an alternative embodiment, a time interval between the first administration time of the therapeutically effective amount of the CpG oligonucleotide and the therapeutically effective amount of R848, and the first administration time of the therapeutically effective amount of the platinum-based chemotherapeutic drug is 1-3 days.

Preferably, a time interval between the first administration time of the therapeutically effective amount of the CpG oligonucleotide and the therapeutically effective amount of R848, and the first administration time of the therapeutically effective amount of the platinum-based chemotherapeutic drug is 2 days.

In an alternative embodiment, a time interval for each administration of the first to fourth administrations of the therapeutically effective amount of the CpG oligonucleotide and the therapeutically effective amount of R848 is 1-7 days.

In an alternative embodiment, the administration cycle for the subject is 1-3 times, and an interval for each administration cycle is 5-7 days.

In an alternative embodiment, a mode of administration of the combination therapy pharmaceutical composition comprises one or more of subcutaneous administration near a tumor and intratumoral injection administration.

In an alternative embodiment, the combination therapy pharmaceutical composition further comprises an additional tumor therapeutic drug; preferably, the additional tumor therapeutic drug is an immunotherapeutic drug, and further preferably, the immunotherapeutic drug is a PD-1/PD-L1 inhibitor.

The present invention also aims to provide a kit or a pharmaceutical combination for treating a tumor or cancer, which comprises a therapeutically effective amount of a platinum-based chemotherapeutic drug, a therapeutically effective amount of a CpG oligonucleotide, and a therapeutically effective amount of R848; preferably, the platinum-based chemotherapeutic drug is selected from one or more of cisplatin, carboplatin, nedaplatin, oxaliplatin, and lobaplatin; preferably, the platinum is cisplatin; the therapeutically effective amount of the CpG oligonucleotide (CpG ODN) is a B-class CpG ODN or a C-class CpG ODN;
more preferably, the B-class CpG ODN is selected from one or more of ODN 1826, ODN 1018, and ODN 2006/7909;
more preferably, the c-class CpG ODN is selected from one or more of ODN M362, ODN 2395, and D-SL03;
the therapeutically effective amount of R848 is selected from one or more of water-soluble R848 and lipid-soluble R848.

Optionally, instructions for use of the medicament are also included.

Specifically, the therapeutically effective amount of the platinum-based chemotherapeutic drug, the therapeutically effective amount of the CpG oligonucleotide, and the therapeutically effective amount of R848 are packaged separately, or the therapeutically effective amount of the platinum-based chemotherapeutic drug is packaged individually, and the therapeutically effective amount of the CpG oligonucleotide and the therapeutically effective amount of R848 are packaged in a mixed manner;
preferably, the therapeutically effective amount of the platinum-based chemotherapeutic drug, the therapeutically effective amount of the CpG oligonucleotide, and the therapeutically effective amount of R848 are sequentially administered to a subject in need thereof;
more preferably, the administrations of the therapeutically effective amount of the CpG oligonucleotide and the therapeutically effective amount of R848 are performed simultaneously, and the administration of the therapeutically effective amount of the platinum-based chemotherapeutic drug is performed prior to the administrations of the therapeutically effective amount of the CpG oligonucleotide and the therapeutically effective amount of R848;
more preferably, the therapeutically effective amount of the platinum-based chemotherapeutic drug is administered 1 time, and the therapeutically effective amount of the CpG oligonucleotide and the therapeutically effective amount of R848 are administered 1-5 times for 1 administration cycle; a time interval between the first administration time of the therapeutically effective amount of the CpG oligonucleotide and the therapeutically effective amount of R848, and the first administration time of the therapeutically effective amount of the platinum-based chemotherapeutic drug is 1-5 days;
preferably, a time interval between the first administration time of the therapeutically effective amount of the CpG oligonucleotide and the therapeutically effective amount of R848, and the first administration time of the therapeutically effective amount of the platinum-based chemotherapeutic drug is 2 days;
a time interval for each administration of the first to fifth administrations of the therapeutically effective amount of the CpG oligonucleotide and the therapeutically effective amount of R848 is 1-14 days;
the administration cycle for administering the therapeutically effective amount of the CpG oligonucleotide and the therapeutically effective amount of R848 to the subject is 1-3 times, and an interval for each administration cycle is 5-7 days.

Further preferably, the kit or the pharmaceutical combination further comprises an additional tumor therapeutic drug; preferably, the additional tumor therapeutic drug is an immunotherapeutic drug, and further preferably, the immunotherapeutic drug is a PD-1/PD-L1 inhibitor.

The fourth objective of the present invention is to provide use of the above composition or kit or pharmaceutical combination in the manufacture of a medicament for treating a tumor or cancer.

In an alternative embodiment, the tumor or cancer is selected from breast cancer, melanoma, liver cancer, basal cell carcinoma, cutaneous squamous cell carcinoma, cutaneous T-cell lymphoma, and colorectal cancer; preferably, the tumor or cancer is selected from breast cancer and melanoma.

The fifth objective of the present invention is to provide a method for treating a tumor or cancer, which comprises administering to a subject a therapeutically effective amount of a platinum-based chemotherapeutic drug, a therapeutically effective amount of a CpG oligonucleotide, and a therapeutically effective amount of R848;
preferably, the platinum-based chemotherapeutic drug is selected from one or more of cisplatin, carboplatin, nedaplatin, oxaliplatin, and lobaplatin; preferably, the platinum is cisplatin; the therapeutically effective amount of the CpG oligonucleotide (CpG ODN) is a B-class CpG ODN or a C-class CpG ODN;
more preferably, the B-class CpG ODN is selected from one or more of ODN 1826, ODN 1018, and ODN 2006/7909;
more preferably, the C-class CpG ODN is selected from one or more of ODN M362, ODN 2395, and D-SL03; the therapeutically effective amount of R848 is selected from one or more of water-soluble R848 and lipid-soluble R848.

In an alternative embodiment, the therapeutically effective amount of the platinum-based chemotherapeutic drug is administered at a dose of 2 μg/kg to 4000μg/kg;
the therapeutically effective amount of the CpG oligonucleotide is administered at a dose of 0.001 mg/time to 32 mg/time;
the therapeutically effective amount of R848 is administered at a dose of 0.001 mg/time to 5 mg/time.

In an alternative embodiment, the therapeutically effective amount of the platinum-based chemotherapeutic drug is administered at a dose of 2 μg/kg to 20 μg/kg;
the therapeutically effective amount of the CpG oligonucleotide is administered at a dose of 0.001 mg/time to 32 mg/time;
the therapeutically effective amount of R848 is administered at a dose of 0.001 mg/time to 1 mg/time.

In a preferred embodiment, the therapeutically effective amount of the platinum-based chemotherapeutic drug is administered at a dose of 2 μg/kg to 400 μg/kg;
the therapeutically effective amount of the CpG oligonucleotide is administered at a dose of 0.05 mg/time to 2 mg/time;
the therapeutically effective amount of R848 is administered at a dose of 0.01 mg/time to 1 mg/time.

In an alternative embodiment, the therapeutically effective amount of the platinum-based chemotherapeutic drug, the therapeutically effective amount of the CpG oligonucleotide, and the therapeutically effective amount of R848 are packaged separately, or the therapeutically effective amount of the platinum-based chemotherapeutic drug is packaged individually, and the therapeutically effective amount of the CpG oligonucleotide and the therapeutically effective amount of R848 are packaged in a mixed manner.

In an alternative embodiment, the therapeutically effective amount of the platinum-based chemotherapeutic drug, the therapeutically effective amount of the CpG oligonucleotide, and the therapeutically effective amount of R848 are sequentially administered to a subject in need thereof;
more preferably, the administrations of the therapeutically effective amount of the CpG oligonucleotide and the therapeutically effective amount of R848 are performed simultaneously, and the administration of the therapeutically effective amount of the platinum-based chemotherapeutic drug is performed prior to the administrations of the therapeutically effective amount of the CpG oligonucleotide and the therapeutically effective amount of R848;
more preferably, the therapeutically effective amount of the platinum-based chemotherapeutic drug is administered 1 time, and the therapeutically effective amount of the CpG oligonucleotide and the therapeutically effective amount of R848 are administered 1-5 times for 1 administration cycle; a time interval between the first administration time of the therapeutically effective amount of the CpG oligonucleotide and the therapeutically effective amount of R848, and the first administration time of the therapeutically effective amount of the platinum-based chemotherapeutic drug is 1-5 days;
preferably, a time interval between the first administration time of the therapeutically effective amount of the CpG oligonucleotide and the therapeutically effective amount of R848, and the first administration time of the therapeutically effective amount of the platinum-based chemotherapeutic drug is 2 days;
a time interval for each administration of the first to fifth administrations of the therapeutically effective amount of the CpG oligonucleotide and the therapeutically effective amount of R848 is 1-14 days;
the administration cycle for administering the therapeutically effective amount of the CpG oligonucleotide and the therapeutically effective amount of R848 to the subject is 1-3 times, and an interval for each administration cycle is 5-7 days.

In an alternative embodiment, the tumor or cancer is selected from breast cancer, melanoma, liver cancer, basal cell carcinoma, cutaneous squamous cell carcinoma, cutaneous T-cell lymphoma, and colorectal cancer; preferably, the tumor or cancer is selected from breast cancer and melanoma.

Compared with the prior art, the present invention has the following beneficial effects:

The present invention provides a combination therapy pharmaceutical composition and a combination therapy method for treating a tumor, wherein the combination therapy pharmaceutical composition is a low-dose platinum-based chemotherapeutic drug combined with CpG and R848; the combination therapy method is to use the low-dose platinum-based chemotherapeutic drug, CpG, and R848 at specific times. The action mechanism of the combination therapy pharmaceutical composition and the combination therapy method for treating the tumor provided by the present invention is as follows: firstly, a low-dose chemotherapeutic drug is used to kill a tumor so as to release a tumor neoantigen, enabling the immune system of an organism to autonomously identify and screen the neoantigen, and an innate and adaptive anti-tumor immune response is then stimulated by using a Toll-like receptor activator, that is, CpG oligonucleotide and R848 immune activators are used to induce and promote the organism to generate an immune response specific to the tumor neoantigen, such that the anti-tumor function of T cells is improved and strengthened, thereby achieving the effect of broadly inhibiting the growth of tumors.

Specifically, the combination therapy of the present invention has the following advantages compared with other existing therapies: (1) it significantly reduces the dosage of chemotherapeutic drugs, reduces the side effects caused by chemotherapy in patients, and improves compliance; (2) it is not limited by the type of tumors and continuous mutations of tumor antigens, the tumor antigens are released by small-dose chemotherapeutic drugs, and the TLR activators activate the internal anti-tumor immunity of the organism to response to the tumor mutations at any time; (3) it has a wide application range, which can be applied to tumor patients without surgical indications, is suitable for new adjuvant therapy to increase the chances of surgical treatment for the patients, can be applied to patients with advanced malignant tumors for whom other treatment methods are ineffective, and can also be applied to postoperative adjuvant therapy, and the application range and prospects of the combination therapy can be superior to the existing products or means of passive immunotherapy or immune checkpoint blockade therapy; and (4) the price is low, thereby significantly reducing the economic burden of the patients and family members, and reducing the national medical insurance burden.

### BRIEF DESCRIPTION OF THE DRAWINGS

In order to more clearly illustrate the technical solutions in the embodiments of the present invention or in the prior art, the drawings required to be used in the description of the embodiments or the prior art are briefly introduced below.

FIG. 1 shows the results of the 4T1 tumor growth curves provided in Example 1 of the present invention;

FIG. 2 is a graph of each group of separated solid tumors provided in Example 1 of the present invention;

FIG. 3 shows the results of each group of tumor weights provided in Example 1 of the present invention;

FIG. 4 shows the results of the phenotype detection of infiltrating lymphocytes in tumor tissues provided in Example 2 of the present invention;

FIG. 5 shows the results of the phenotype detection of immune cells in draining lymph nodes provided in Example 2 of the present invention;

FIG. 6 shows the effect of different time points of administration on the 4T1 tumor growth curve provided in Example 3 of the present invention;

FIG. 7 shows tumor inoculation and administration sites and shows the results of tumor growth curves provided in Example 4 of the present invention;

FIG. 8 shows the results of the B16 tumor growth curves provided in Example 5 of the present invention;

FIG. 9 shows the results of in vitro stimulation of mouse spleen cells to secrete TNF-α cytokines by CpG and R848 from different sources provided in Example 6 of the present invention;

FIG. 10 shows the results of tumor growth curves inhibited by CpG and R848 from different sources provided in Example 6 of the present invention;

FIG. 11 shows the body weight results of mice provided in Example 7 of the present invention;

FIG. 12 shows the results of the 4T1 tumor volume growth curves provided in Example 7 of the present invention;

FIG. 13 shows the results of each group of tumor weights provided in Example 7 of the present invention;

FIG. 14 is a graph of each group of separated solid tumors provided in Example 7 of the present invention;

FIG. 15 shows the results of expression of Ly6C monocytes in draining lymph nodes and expression of IFNγ, TNFα, perforin, and GzmB in CD4⁺ and CD8⁺ T cells provided in Example 8 of the present invention;

FIG. 16 shows the secretion levels of IFNγ, IFNα, and TNFα in the serum of each group of mice provided in Example 8 of the present invention;

FIG. 17 shows the quantitative results and immunofluorescence labeling results of infiltrating T cells and B cells in tumor tissues provided in Example 8 of the present invention;

FIG. 18 shows the results of volume growth curves of each group of B16 tumors provided in Example 9 of the present invention;

FIG. 19 is a graph of each group of separated solid tumors provided in Example 9 of the present invention;

FIG. 20 shows the results of each group of tumor weights provided in Example 9 of the present invention;

FIG. 21 shows the body weight results of each group of mice provided in Example 9 of the present invention;

FIG. 22 shows the weight results of important organs of each group of mice provided in Example 9 of the present invention;

FIG. 23 is a flowchart of administration to animals provided in Example 10 of the present invention;

FIG. 24 shows the body weight changes of mice during administration provided in Example 10 of the present invention;

FIG. 25 shows the tumor volume growth curves of mice during administration provided in Example 10 of the present invention;

FIG. 26 shows the survival curves of tumor-bearing mice during administration provided in Example 10 of the present invention;

FIG. 27 shows the body weight changes of mice during administration provided in Example 11 of the present invention;

FIG. 28 shows the tumor volume growth curves of mice during administration provided in Example 11 of the present invention;

FIG. 29 shows the survival curves of tumor-bearing mice during administration provided in Example 11 of the present invention;

FIG. 30 shows the body weight changes of mice during administration provided in Example 12 of the present invention;

FIG. 31 shows the tumor volume growth curves of mice during administration provided in Example 12 of the present invention;

FIG. 32 shows the survival curves of tumor-bearing mice during administration provided in Example 12 of the present invention;

FIG. 33 shows the body weight changes of mice during administration provided in Example 13 of the present invention;

FIG. 34 shows the tumor volume growth curves of mice during administration provided in Example 13 of the present invention;

FIG. 35 shows the survival curves of tumor-bearing mice during administration provided in Example 13 of the present invention;

FIG. 36 shows the results of the 4T1 tumor volume growth curves provided in Example 14 of the present invention.

### DETAILED DESCRIPTION

The technical solutions of the present invention will be described clearly and completely with reference to the following examples, and it should be apparent that the described examples are some of the examples of the present invention but not all of them. Based on the examples of the present invention, all other examples obtained by those of ordinary skills in the art without creative work shall fall within the protection scope of the present invention.

The technical solutions and beneficial effects of the present invention will be further described below with reference to preferred examples.

### Example 1: Tumor Inhibitory Effect of Low-Dose Cisplatin Combined with CpG and R848 Adjuvants

Balb/c mice were randomly grouped. The fur of each mouse was shaved using a mouse shaver at the posterior dorsal, proximal to the hip position, on the right side. The tumor model was constructed by subcutaneously injecting a 4T1 breast cancer cell suspension (3×10⁵ cells/mouse) at the posterior dorsal, proximal to the hip position, on the right side according to 100 μL/mouse. After tumors grew to a suitable size, about 10 mm², and formed about 3 days after inoculation, the mice were given drugs according to a PBS control group, cisplatin (CDDP) control group, CpG+R848 (CR) adjuvant single administration group, and CDDP combined with CR adjuvant administration group.

The specific mouse administration groups were as follows: (1) CDDP 2 μg (2 g/kg cisplatin), (2) CDDP 5 μg (5 μg/kg cisplatin), (3) CDDP 2 μg+CR 10 μg (2 μg/kg cisplatin, 10 μg/mouse CpG, and 10 μg/mouse R848), (4) CDDP 5 μg+CR 10 μg (5 μg/kg cisplatin, 10 μg/mouse CpG, and 10 μg/mouse R848), (5) CDDP 2 μg+CR 50 μg (2 μg/kg cisplatin, 50 μg/mouse CpG, and 50 μg/mouse R848), (6) CR 10 μg (10 μg/mouse CpG and 10 μg/mouse R848), (7) CR 50 μg (50 μg/mouse CpG and 50 μg/mouse R848), and (8) a PBS negative control group. Among them, CpG is called CpG oligonucleotide (CpG ODN).

Drug preparation: CDDP was dissolved in PBS and prepared at a concentration corresponding to the required administration dose in a volume of 100 μL per mouse. R848 was dissolved in a specialized water-soluble vehicle to prepare a 10 mg/mL stock solution; 0.1 mL of the stock solution was taken for each administration, the volume was brought to 1 mL, and a 1 mg/mL solution was prepared; the CpG used in this experiment was ODN 1826 (CpG1826), which was prepared at 1 mg/mL with PBS before each administration. When the administration was performed, 1 mg/mL CpG1826 solution and R848 solution were mixed in equal volumes according to the required amount to form different combinations of “CR” mixed adjuvants, and 100 μL of the “CR” mixed adjuvant was administered to each mouse. CDDP is a product from Dalian Meilun Biotech Co., Ltd., with Catalog No. MB1055; R848 is a product from InvivoGen, with Catalog No. tlrl-r848-5; and CpG1826 was purchased from Zixi Biotechnology Co., Ltd., with Catalog No. NS-004984-001.

Administration time: The day of tumor inoculation was defined as day 0. The CDDP group and the CR adjuvant combined with CDDP administration group were given 100 μL of CDDP on day 3, and the PBS group and the CR group were given 100 μL of PBS on day 3. The CR adjuvant group and the CR adjuvant combined with CDDP administration group were given 100 μL of the CR mixed drug on day 5, day 12, and day 19, and the PBS control group and the CDDP control group were given 100 μL of PBS at the corresponding time points. The method and sites of administration were subcutaneous injection near the tumor. On day 4 after the last administration, the mice were sacrificed.

Tumor size measurement: The tumor volume was measured 2 times a week using a vernier caliper, the long and short diameters of the tumor were measured, and the tumor volume calculation formula was: tumor volume=0.5×long diameter×short diameter² (mm³). The tumor area calculation formula was: tumor size =long diameter×short diameter (mm²).

Tumor weighing: At the end of the experiment, tumor tissues of the mice were separated and weighed using an analytical balance; the weights were recorded, and the tumors were photographed for recording.

Conclusion: The tumor size growth curves of mice in each group of administration groups are shown in FIG. 1. With the increase of days, it was found that the tumors in the PBS control group grew rapidly, while the tumors in the mice in the 5th group of low-dose CDDP combined with high-dose CR adjuvant administration group (CDDP 2 μg+CR 50 μg group) grew slowest, the growth and proliferation of cells were inhibited significantly, and the effect of inhibiting the tumor growth was optimal compared with the other groups.

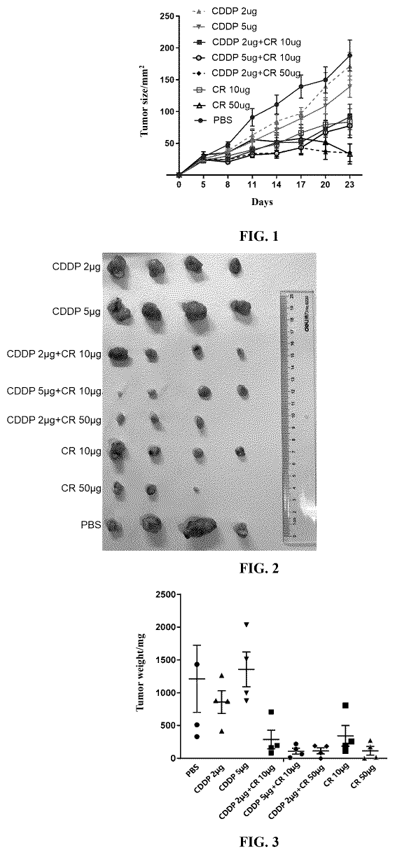

Solid tumors separated from each group of mice are shown in FIG. 2, and the corresponding tumor weight results are shown in FIG. 3. It was found that the tumors grown from the groups of high-dose CDDP combined with low-dose CR adjuvant administration group (CDDP 5 μg+CR 10 μg group), low-dose CDDP combined with high-dose CR adjuvant administration group (CDDP 2 μg+CR 50 μg group), and high-dose CR adjuvant single administration group (CR 50 μg group) were the lightest relative to the other groups.

### Example 2: Flow Cytometry Analysis of Immune Cell Phenotype in Tumor Tissues and Draining Lymph Nodes

Experimental groups were the same as those in Example 1, and an additional group of blank naive mouse group without tumor inoculation and drug administration was added. The construction of a 4T1 breast cancer cell tumor model, drug preparation, and time and method of administration were the same as those in Example 1. The mice were sacrificed on day 7 after the last administration, tumors and lymph nodes were collected, and infiltrating lymphocytes (TIL) CD8⁺ T cells in tumor tissues and CD4⁺ T cells in draining lymph nodes (dLNs) were detected by flow cytometry.

Tumor tissue samples: After the mice were sacrificed by cervical dislocation, tumors of the mice were collected and weighed. The tumor tissues were minced to millet grain size, and 5.95 mL of RPMI1640 medium containing 0.48 mg of collagenase solution and 5 μL of 1000 U/mL DNase I were added, followed by incubation and digestion at 37° C. for 45 min. After the digestion was completed, 3 mL of complete medium was added to terminate the digestion process. After filtration, the medium was supplemented to 10 mL, and the resulting mixture was centrifuged in a 15 mL centrifuge tube at 400 g for 5 min. Then, the samples were resuspended in a flow staining mixture for staining, stained with a fixable viability dye for 15 min, and washed. The samples were then stained with anti-mouse CD3, CD8a, CD223 (LAG3), CD366 (TIM3), and CD279 (PD-1) antibodies for 15 min in the dark at room temperature, and 150 μL of PBS solution was added for termination. The resulting mixtures were centrifuged, the supernatants were discarded, and the samples were resuspended and tested on a machine. The samples were detected by LSRFortessa, and the acquired flow cytometry data needed to be analyzed on the Flowjo software.

Lymph node samples: After the mice were sacrificed by cervical dislocation, the lymph nodes of the mice were taken and placed in a 1.5 mL EP centrifuge tube, 300 μL of PBS solution was added, and the resulting mixture was ground with a grinding pestle. After filtration, centrifugation was performed. The lymph node samples were directly added to the PBS solution for resuspension. The samples were stained with the fixable viability dye for 15 min and washed. The samples were then stained with anti-mouse CD3, CD4, CD223 (LAG3), and CD279 (PD-1) antibodies in the dark at room temperature for 15 min and washed. The cells were permeabilized with fixation/permeabilization buffer for 1 h, and intracellular staining was performed with anti-mouse Foxp3 at room temperature for 60 min. After centrifugation, the samples were resuspended in the PBS buffer and tested on the machine. The samples were detected by LSRFortessa, and the acquired flow cytometry data needed to be analyzed on the Flowjo software.

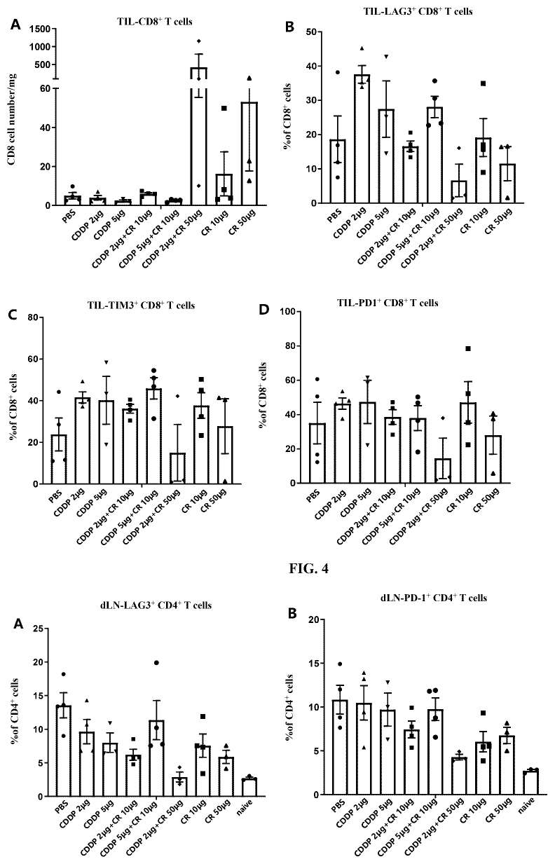

Conclusion: The detection results of tumor tissue samples are shown in FIG. 4. The results in FIG. 4A show that the number of infiltrating CD8⁺ T cells in the tumor tissues of the low-dose CDDP combined with high-dose CR adjuvant administration group (CDDP 2 μg+CR 50 μg group) was the highest, which was significantly superior to other groups, indicating that this group has an excellent effect in activating CD8⁺ T cell immune responses, inducing CD8⁺ T cells to play an immune killing role, thereby inhibiting the tumor growth. LAG3, TIM3, PD-1, and Foxp3 are related inhibitory molecules that induce T cell depletion. The results in FIGS. 4B-4C show that the number of LAG3⁺ CD8⁺ T cells, TIM3⁺ CD8⁺ T cells, and PD-1+ CD8⁺ T cells in the tumor tissues of the low-dose CDDP combined with high-dose CR adjuvant administration group (CDDP 2 μg+CR 50 μg group) was significantly lower than that of other groups, indicating that this group can prevent the depletion of CD8⁺ T cells to a certain extent, allowing more CD8⁺ T cells to exert effector functions, thereby inhibiting the tumor growth.

The detection results of lymph node samples are shown in FIG. 5. The results in FIGS. 5A-5C show that the number of LAG3⁺ CD4⁺ T cells, PD-1⁺ CD4⁺ T cells, and Foxp3⁺ CD4⁺ T cells in the lymph node tissues of the low-dose CDDP combined with high-dose CR adjuvant administration group (CDDP 2 μg+CR 50 μg group) was significantly lower than that of other tumor-inoculated administration groups, and the level of this group was comparable to that of the blank naive mice without tumor inoculation and drug administration, indicating that the expression of inhibitory molecules related to CD4⁺ T cell depletion induced in this group and the number of Treg cells were the lowest, which demonstrates that this group can prevent the depletion of CD4⁺ T cells to a certain extent, allowing more CD4⁺ T cells to exert effector functions, thereby inhibiting the tumor growth.

Based on the combined results of Examples 1-2, it can be reasonably inferred that the low-dose CDDP combined with high-dose CR adjuvant administration group (CDDP 2 μg+CR 50 μg group) has the best therapeutic effect on tumors and is a regimen that can be used in subsequent clinical studies.

### Example 3: Exploration of Administration Time of CDDP Combined With CpG and R848 to Inhibit Tumor Growth

The above experiments explored the optimal dose range for the administration of CDDP combined with CpG and R848, and this example mainly explored whether the effect of tumor growth inhibition is affected by the difference in the time of administration. The construction of a 4T1 breast cancer cell tumor model, drug preparation, and tumor size measurement were the same as those in Example 1. Experimental administration groups were as follows: (1) a PBS negative control group, (2) CR (10 μg/mouse CpG and 10 μg/mouse R848), (3) CDDP (2 μg/kg cisplatin), (4) CDDP+CR (administration time 1) (2 μg/kg cisplatin, 10 μg/mouse CpG, and 10 μg/mouse R848), and (5) CDDP+CR (administration time 2) (2 μg/kg cisplatin, 10 μg/mouse CpG, and 10 μg/mouse R848).

Administration time: The day of tumor inoculation was defined as day 0, and the method and sites of administration were subcutaneous injection near the tumor.
(1) PBS group: 100 μL of PBS was administered on day 5, day 12, and day 19.
(2) CR group: 100 μL of CR mixed drug was administered on day 5, day 12, and day 19.
(3) CDDP group: 100 μL of CDDP was administered on day 5, and 100 μL of PBS was administered on day 12 and day 19.
(4) CDDP+CR (administration time 1): 100 μL of CDDP and 100 μL of CR mixed drug were administered on day 5, and 100 μL of CR mixed drug was administered on day 12 and day 19.
(5) CDDP+CR (administration time 2, same as that in Example 1): 100 μL of CDDP was administered on day 3, and 100 μL of CR mixed drug was administered on day 5, day 12, and day 19.

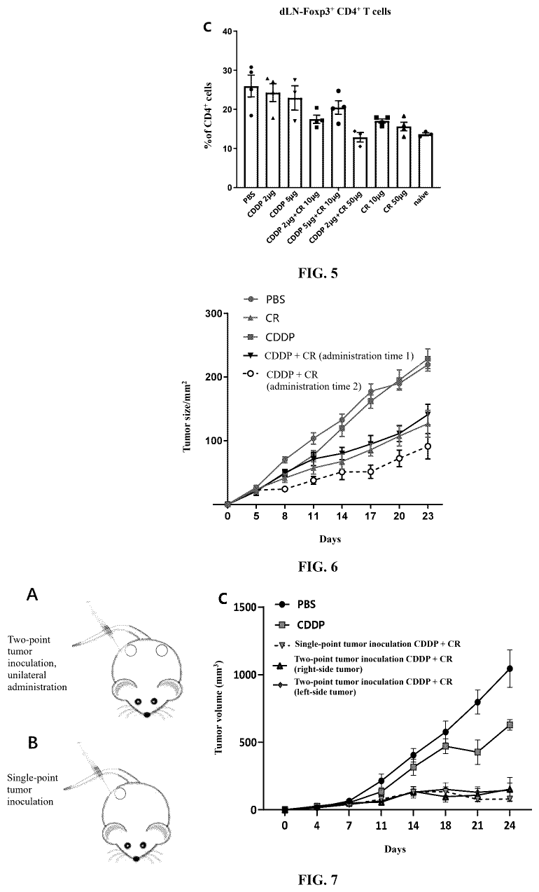

Conclusion: Tumor size growth curves are shown in FIG. 6. With the increase of days, the tumors in the PBS control group grew rapidly, and the tumors grew slowest in the CDDP+CR (administration time 2) administration group compared with the other groups, which was significantly superior to the CDDP+CR (administration time 1) administration group, indicating that the administration method of the “administration time 2” provided by the present invention has a better effect on inhibiting the tumor growth.

### Example 4: Inhibitory Effect of CDDP Combined With CpG and R848 on Tumors at Distal Sites of Administration

The above experiments set a single-point tumor inoculation, and the administration site was subcutaneous injection near the tumor. It was demonstrated that CDDP combined with CpG and R848 had a significant inhibitory effect on tumors at the proximal sites of administration. To observe whether CDDP combined with CpG and R848 could have the same inhibitory effect on tumors at the distal sites, 3×10⁵ of 4T1 tumor cells were subcutaneously inoculated on both sides of the back of mice proximal to the hip position, and CDDP, CpG, and R848 were sequentially administered near the tumor on the right side only (FIG. 7A, two-point tumor inoculation, only right-side administration); a single-point tumor inoculation group was simultaneously set, i.e., 3×10⁵ of 4T1 cells were inoculated on the right side and administered subcutaneously near the tumor on the right side (FIG. 7B, single-point tumor inoculation, right-side administration). The administration time and the tumor measurement method were the same as those in Example 1. Specific experimental administration groups were as follows: (1) a PBS negative control group, (2) CDDP (2 g/kg cisplatin), (3) single-point tumor inoculation CDDP+CR (2 μg/kg cisplatin, 50 μg/mouse CpG, and 50 μg/mouse R848), and (4) two-point tumor inoculation CDDP+CR (2 μg/kg cisplatin, 50 ug/mouse CpG, and 50 μg/mouse R848).

Conclusion: The experimental results are shown in FIG. 7C. With the increase of days, the tumors in the PBS control group grew rapidly, while the growth of the tumors in the single-point tumor inoculation CDDP+CR administration group, the tumors on the right side in the two-point tumor inoculation CDDP+CR administration group, and the tumors on the left side in the two-point tumor inoculation CDDP+CR administration group were significantly inhibited, with the slowest tumor growth and almost overlapping tumor growth curves, indicating that when two-point tumor inoculation was performed and the drug was only administered on the right side, i.e., the single-side administration was performed, CDDP combined with CpG and R848 could not only inhibit the growth of the tumor on the right side, but also significantly inhibit the growth of the tumor on the left side, which demonstrates that CDDP combined with CpG and R848 can significantly inhibit the growth of proximal tumors, but also significantly inhibit the growth of distal tumors, indicating that this combination therapy composition and the combination therapy method thereof have inhibitory effects on systemic tumor lesions.

### Example 5: Tumor Inhibitory Effect of Low-dose Cisplatin Combined With CpG and R848 Adjuvants on Melanoma

In order to further explore whether the low-dose CDDP combined with CpG and R848 adjuvants has the same tumor inhibitory effect on melanoma, a mouse melanoma B16-F10 tumor model was constructed, and the tumor model was constructed according to the number of tumor cells of 3×10⁵ cells/mouse. The drug preparation, time and method of administration, and tumor size measurement were the same as those in Example 1. Experimental administration groups were as follows: (1) a PBS negative control group, (2) CDDP 2 μg (2 μg/kg cisplatin), (3) CR 50 μg (50 μg/mouse CpG and 50 μg/mouse R848), and (4) CDDP 2 μg+CR 50 μg (2 μg/kg cisplatin, 50 μg/mouse CpG, and 50 μg/mouse R848).

Conclusion: The tumor size growth curves of melanoma are shown in FIG. 8. With the increase of days, it was found that the tumors in the PBS control group grew rapidly, while the tumors of mice in the 4th group of CDDP 2 μg+CR 50 μg administration group grew slowest, the growth and proliferation of cells were inhibited significantly, and the effect of inhibiting the tumor growth was optimal compared with other groups. It was indicated that the low-dose cisplatin combined with CpG and R848 adjuvants (CDDP 2 μg+CR 50 μg group) provided by the present invention not only had a significant tumor inhibitory effect on breast cancer cells, but also had a significant tumor inhibitory effect on melanoma, suggesting that the low-dose cisplatin combined with CpG and R848 adjuvants provided by the present invention has an effect of broadly inhibiting the tumor growth.

### Example 6: Comparison of Effects of CpG or R848 From Different Sources

Due to the fact that CpG and R848 used in all the above examples are research-grade reagents, in order to promote the clinical application of the present invention, it is necessary to use pharmaceutical grade reagents. Therefore, we performed experiments to compare if the effects of the research-grade reagents CpG1826 and R848 (water soluble) and the pharmaceutical reagents CpG1018 (ODN 1018) and R848 (lipid soluble) would be the same. CpG1018 was purchased from Zixi Biotechnology Co., Ltd., with Catalog No. NS-007772-001, and R848 (lipid soluble) was purchased from Hubei Widely Chemical Technology Co., Ltd., with Catalog No. 144875-48-9.

Drug preparation: The preparation and use of the research-grade reagents CpG1826 and R848 (water soluble) were the same as those in Example 1; the pharmaceutical agent R848 (lipid soluble) was dissolved in DMSO to prepare a 10 mg/mL stock solution, 0.1 mL of the stock solution was taken for each administration, the volume was brought to 1 mL, and a 1 mg/mL solution was prepared; CpG1018 was prepared at 1 mg/ml with PBS before each administration.

### 1. In Vitro Experiments

The spleens of three blank BABL/c mice were added to a 30 mm cell culture dish, 1640 medium was added, and the resulting mixture was ground, filtered through a copper screen, and centrifuged at 1500 rpm for 3 min. The supernatant was discarded, 2 mL of a red blood cell lysis buffer was added, and the samples were left to stand for 2 min, terminated with 4 mL of 1640+10% FBS, and centrifuged at 1500 rpm for 3 min. The supernatant was discarded, the samples were resuspended in 1640+10% FBS, and the cells were counted. The cells were plated in a 96-well cell culture plate at 2×10⁶ cells/well, with 200 μL of culture medium per well. Various doses of CpG and R848 were added, with LPS as a positive control and PBS-only blank wells as a negative control (NC). After the cells were stimulated for 18-20 h, the cell culture supernatant was collected, and the expression of cytokines in each 10⁶ mouse spleen cells was determined by using a mouse TNF-α detection kit (purchased from NeoBioscience Technology Co., Ltd., Catalog No. EMC102a.96) according to the instructions.

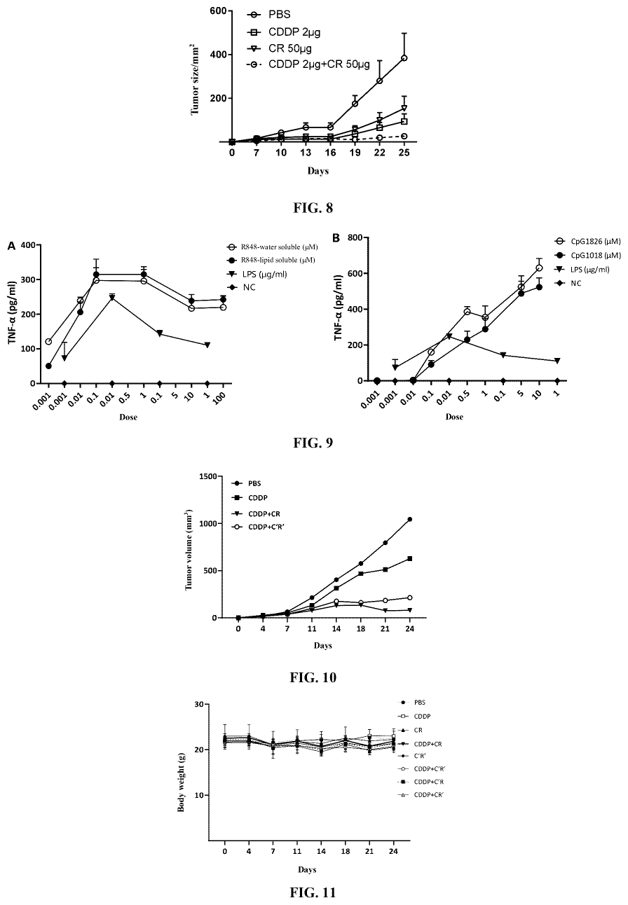

Conclusion: The experimental results are shown in FIG. 9. The results in FIG. 9A show that the levels of TNF-α cytokines produced from BALB/c mouse spleen cells stimulated in vitro by research-grade R848 (water soluble) and pharmaceutical grade R848 (lipid soluble) were comparable. The results in FIG. 9B also show that the levels of TNF-α cytokines produced from BALB/c mouse spleen cells stimulated in vitro by research-grade CpG1826 and pharmaceutical grade CpG1018 were comparable. It was indicated that the type and source of CpG and R848 were changed, but the levels of cytokines produced from mouse spleen cells were not significantly influenced, demonstrating that the pharmaceutical grade CpG and R848 have the same effects as the research-grade reagents.

### 2. In Vivo Experiments

The construction of a 4T1 breast cancer cell tumor model, time and method of administration, and tumor size measurement were the same as those in Example 1. Experimental administration groups were as follows: (1) a PBS negative control group, (2) CDDP (2 μg/kg cisplatin), (3) CDDP+CR (2μg/kg cisplatin, 50 μg/mouse CpG1826, and 50 μg/mouse R848 (water soluble)), and (4) CDDP+C′R′ (2 μg/kg cisplatin, 50 μg/mouse CpG1018, and 50 μg/mouse R848 (lipid soluble)).

Conclusion: The experimental results are shown in FIG. 10. The tumor growth in mice was slowest in the CDDP+CR administration group and the CDDP+C′R′ administration group, the growth and proliferation of cells were significantly inhibited, and the tumor growth inhibitory effects were comparable between the two groups, further demonstrating that the pharmaceutical grade CpG1018 and R848 (lipid soluble) have the same tumor inhibitory effects as the research-grade reagents, laying a foundation for their clinical application.

### Example 7: Inhibitory Effects of Cisplatin Combined with CpG and R848 Adjuvants with Different Formulas on 4T1 Breast Cancer Tumors

To further explore the tumor inhibitory effects after modifying the formulas, the experimental administration groups were as follows, 6 mice per group: (1) a PBS negative control group, (2) CDDP (2 g/kg cisplatin), (3) CR (50 μg/mouse CpG1826 and 50 μg/mouse water-soluble R848), (4) CDDP+CR (2 μg/kg cisplatin, 50 μg/mouse CpG1826, and 50 μg/mouse water-soluble R848), (5) C′R′ (50 μg/mouse CpG1018 and lipid-soluble 50 μg/mouse R848), (6) CDDP+C′R′ (2 μg/kg cisplatin, 50 μg/mouse CpG1018, and 50 μg/mouse lipid-soluble R848), (7) CDDP+C′R, (2 μg/kg cisplatin, 50 μg/mouse CpG1018, and 50 μg/mouse water-soluble R848), and (8) CDDP+CR′ (2 μg/kg cisplatin, 50 μg/mouse CpG1826, and 50 μg/mouse lipid-soluble R848). The construction of a 4T1 breast cancer cell tumor model, procedure and method of administration, and tumor size measurement were the same as those in Example 1. The preparation of CpG and R848 was the same as that in Example 6.

### 7.1 Mouse Body Weight Measurement

After tumor inoculation, the body weight of the mice was measured once every 3 days or 4 days and the change in injection sites was observed; through observation, the local injection sites of each group of mice had no swelling, rupture, and the like; in addition, as shown in FIG. 11, the body weight of mice in each group was relatively stable in the combination therapy groups, there was a slight downward trend in the body weight after each administration, but no continuous weight loss was observed, and the body weight recovered within 3 days.

### 7.2 Tumor Volume Measurement

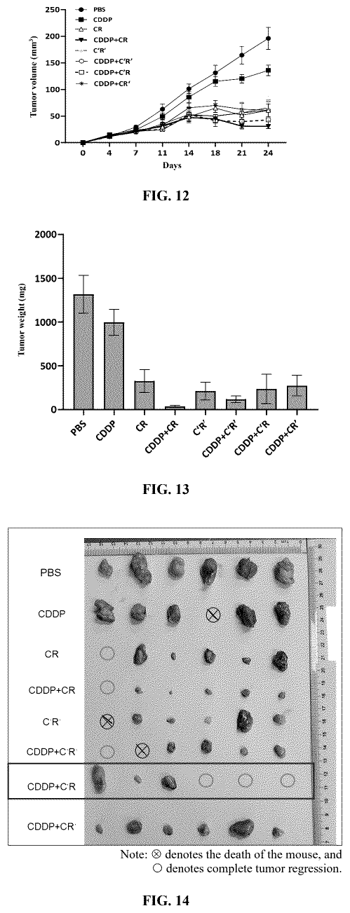

After tumor inoculation, the tumor volume of mice was measured once every 3 days or 4 days. The tumor volume growth curves of the mice in each group are shown in FIG. 12. With the increase of days, the tumors in the PBS control group and the CDDP single administration group grew rapidly, while the growth and proliferation of tumor cells in other combination therapy groups were significantly inhibited, and all the other combination therapy groups exhibited relatively good tumor inhibitory effects. Among these, the tumor inhibitory effects of the CDDP+CR group and the CDDP+C′R group were optimal.

### 7.3 Tumor Weight Weighing and Mouse Survival Recording

At the end of the experiment, tumor tissues of the mice were separated and weighed using an analytical balance; the weights were recorded, and the tumors were photographed for recording. The corresponding tumor weight results are shown in FIG. 13. It was found that the tumors in the PBS control group and the CDDP single administration group grew too large and overweight, the tumor weights of the other groups significantly decreased compared with the two groups, and the tumors in the CDDP+CR group were lightest. The solid tumors separated from each group of mice are shown in FIG. 14. One mouse died during the administration period in each group of the C′R′ group and the CDDP+C′R′ group. In the CR group, the CDDP+CR group, and the CDDP+C′R′ group, one mouse in each group showed complete tumor regression after administration. In the CDDP+C′R group, three mice showed complete tumor regression after administration, and there was no mouse death in the CDDP+C′R group after administration.

According to the experimental results of this example, considering the safety and tumor inhibitory effects, the pharmaceutical composition of CDDP+C′R was selected as the preferred formula, and the tumor adjuvant (C′R) consisting of CpG1018 and water-soluble R848 was named CR108 for the subsequent experiments.

### Example 8: Mechanism Study on Immune Effects of Cisplatin Combined with CR108 Adjuvant

The 4T1 tumor model of breast cancer in mice was constructed with a tumor cell number of 5×10⁵ cells/mouse. The procedure and method of administration were the same as those in Example 7. Experimental administration groups were as follows, 5 mice per group: (1) a PBS negative control group, (2) CDDP (2 μg/kg cisplatin), (3) CDDP+CR108 (2 82 g/kg cisplatin, 50 μg/mouse CpG1018, and 50 μg/mouse water-soluble R848), and (4) CR108 (50 μg/mouse CpG1018 and 50 μg/mouse water-soluble R848).

### 8.1 Flow Cytometry Detection of Immune Cells

The fluorescence-labeled antibodies used were: anti-mouse CD4 (GK1.5), CD8a (53-6.7), CD11b (M1/70), and Ly6C (HK1.4) from Biolegend; anti-mouse perforin (eBioOMAK-D), CD3e (145-2C11), TNFα (MP6-XT22), anti-mouse IFNγ (XMG1.2), Granzyme B (NGZB), and fixable viability dye eFluor 780 from eBioscience. Mice were sacrificed after administration at appropriate time, draining lymph nodes (dLNs) were collected, and a suspension of lymphocytes was prepared. The resulting lymphocytes were washed once with PBS and stained with cell surface antibodies at room temperature for 15 min. When detecting intracellular antigens, it was necessary to permeabilize the cells with a fixation/permeabilization buffer for 1 h after completion of the cell surface antibody staining, and then the cells were stained with intracellular antibodies at room temperature for 1 h. After the cells were washed and resuspended, all stained samples were detected on LSRFortessa (BD Biosciences), and the acquired flow cytometry data needed to be analyzed on the Flowjo software.

Conclusion: On day 3 after the first administration of CR108, the level of immune cells, Ly6C monocytes, in the draining lymph nodes of the CDDP+CR 108 group was significantly increased compared to the other groups (FIG. 15A), indicating that the CDDP+CR108 group has a better ability to enhance the immune system of tumor mice. IFNγ, TNFα, perforins, and granzymes (GzmB) play an important role in the anti-tumor immune system, and the pores formed by perforins can allow granzymes to enter tumor cells to cause apoptosis of the tumor cells. As shown in FIGS. 15B-15G, 3 days after the second administration of CR108, it was found that the percentages of IFNγ⁺ CD4⁺ T cells, TNFα+CD4⁺ T cells, IFNγ⁺CD8⁺ T cells, TNFα⁺CD8⁺ T cells, perforin⁺ CD8⁺ T cells, and GzmB+CD8⁺ T cells in the lymph node tissues of tumor mice in the CDDP+CR108 group were significantly higher than those in other groups, indicating that the CDDP+CR108 administration group resulted in activation of CD8⁺ and CD4⁺ T cells in the draining lymph nodes, thereby promoting a significant increase in IFNγ, TNFα, perforin, and granzyme levels, and thus achieving an excellent anti-tumor purpose.

### 8.2 Cytokine Detection by ELISA

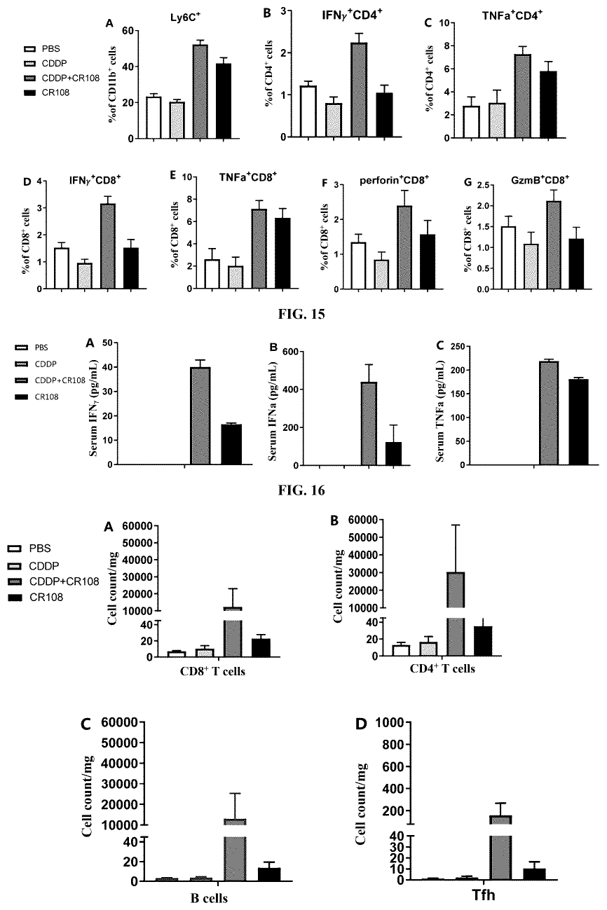

Furthermore, IFNγ, IFNα, and TNFα secreted from the serum of the tumor mice were detected at the same time. Using a multi-cytokine ELISA kit (Multi Sciences) and following the instructions, the concentrations of IFNγ and TNFα in the serum were detected 3 h after the first administration of CR108, and the concentration of the cytokine, IFNα, was detected 4 h after the second administration of CR108. As shown in FIGS. 16A-16C, the secretion levels of IFNγ, IFNα, and TNFα in the CDDP+CR108 treatment group were significantly higher than those in the other groups.

8.3 Detection of infiltrating T cells and B cells in tumor tissues by flow cytometry and fluorescent staining Quantification of CD4⁺ T, CD8⁺ T, B220⁺ B, and PD1⁺ CXCR5⁺ Tfh cells infiltrated in tumor tissues: Tumors of mice were collected on day 7 after the third administration of CR108, and the tumor tissues were cut into small pieces; the small pieces of tumor tissues were incubated with collagenase type IV (Sigma, 80 μg/mL) and DNase I (Sigma) (50 U/mL) for 45 min, followed by mechanical dispersion of the tumor pieces and filtration through a 40 mm sieve; finally, flow cytometry antibody staining and flow cytometry analysis were performed; the flow cytometry antibodies used included CD45 (30-F11), CD4 (GK1.5), CD8a (53-6.7), B220 (RA3-6B2), CXCR5 (L138D7), and PD-1 (RMP1-30).

Immunofluorescence labeling of infiltrating T cells and B cells in tumor tissues: Tumors of mice were collected on day 7 after the third administration of CR108, and the tumor tissues were fixed in 4% paraformaldehyde (PFA)/PBS for over 24 h and then embedded in paraffin. The modified-tissue paraffin blocks were sectioned with a section thickness of 4 um on a paraffin microtome. The primary antibodies used were B220 (RA3-6B2, eBiosciences) and CD3 (GB111337, Servicebio). The secondary antibodies were cyanin 3 Goat anti-rat IgG (Cy3, GB21302, Servicebio) and 488 Goat anti-rabbit IgG (GB25303, Servicebio). The sections were then incubated with TSA (Servicebio) for 10 min in the dark, and then immersed in a citrate repair solution for microwave-based tissue repair, so as to achieve multiple fluorescent staining. The cell nuclei were stained with DAPI (Sigma). The sections were scanned with a Panoramic scanner (3D HITECH) and analyzed with Caseviewer.

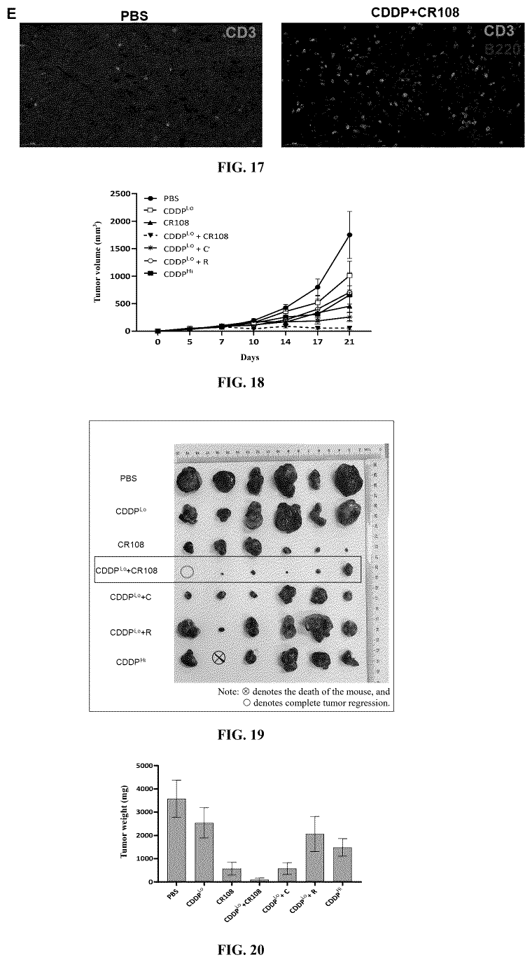

Conclusion: The infiltration extent of immune cells in the tumor tissues of each administration group was further detected. As shown in FIGS. 17A-17D, it was found that the CDDP+CR108 treatment group exhibited a significant increase in the number of CD8⁺ T cells, CD4⁺ T cells, Tfh cells, and B cells in the tumor tissues compared to the other groups, with the largest number of infiltrating cells; additionally, the visual diagram of T cells and B cells infiltrating in the tumor tissues detected by immunofluorescence labeling is shown in FIG. 17E, and it was found that a large number of T cells and B cells were infiltrated in the tumor tissues in the CDDP+CR108 treatment group, further indicating that CDDP+CR108 can more effectively enhance the immune response in tumor mice, promoting the immune cells to enter the tumor tissues and exert anti-tumor effects.

### Example 9: Inhibitory Effect of Cisplatin Combined with CR108 Adjuvant on Mouse Melanoma

This example further explored the effect of cisplatin combined with CR108 adjuvant on mouse melanoma. A B16-F10 mouse melanoma model was constructed by subcutaneously inoculating tumor cells on the right side of the back of the mice proximal to the hip position at 3×10⁵ cells/mouse, and the administration was performed when the tumor volume was about 20 mm³-30 mm³ on day 5 after tumor inoculation.

Specific mouse administration groups were as follows: (1) a PBS negative control group, (2) CDDP^{Lo} (2 μg/kg cisplatin), (3) CR108 (50 μg/mouse CpG1018 and 50 μg/mouse water-soluble R848), (4) CDDP^{Lo}+CR108 (2 μg/kg cisplatin, 50 μg/mouse CpG1018 and 50 μg/mouse water-soluble R848), (5) CDDP^{Lo}+C′ (2 μg/kg cisplatin and 50 μg/mouse CpG1018), (6) CDDP^{Lo}+R (2 μg/kg cisplatin and 50 μg/mouse water-soluble R848), and (7) CDDP^{Hi} (4 mg/kg cisplatin, regular cisplatin chemotherapeutic drug dose). Except for Group (7), which received a high dose of cisplatin by intraperitoneal injection, all other groups were administered by subcutaneous injection near the tumor.

Administration time: The day of tumor inoculation was defined as day 0.
(1) PBS group: 100 μL of PBS was administered on day 5, day 7, day 14, and day 21.
(2) CDDP^{Lo} group: 100 μL of CDDP was administered on day 5, and 100 μL of PBS was administered on day 7, day 14, and day 21.
(3) CR108 group: 100 μL of PBS was administered on day 5, and 100 μL of CR108 mixed drug was administered on day 7, day 14, and day 21.
(4) CDDP^{Lo}+CR108: 100 μL of CDDP was administered on day 5, and 100 μL of CR108 mixed drug was administered on day 7, day 14, and day 21.
(5) CDDP^{Lo}+C′: 100 μL of CDDP was administered on day 5, and 100 μL of CpG was administered on day 7, day 14, and day 21.
(6) CDDP^{Lo}+R: 100 μL of CDDP was administered on day 5, and 100 μL of R848 was administered on day 7, day 14, and day 21.
(7) CDDP^{Hi} group: 100 μL of CDDP was administered on day 7, day 14, and day 21.

### 9.1 Tumor Volume Measurement

The tumor volume growth curve of mice in each group is shown in FIG. 18. With the increase of days, compared with the PBS control group, the growth and proliferation of tumor cells in the CDDP^{Lo}+CR108 administration group were significantly inhibited, the tumor growth was slowest, the volume was minimum, and basically no increase occurred, so that this administration group was significantly superior to other administration groups.

### 9.2 Tumor Weight Weighing and Mouse Survival Recording

On day 4 after the last administration, mice were sacrificed, tumor tissues of the mice were separated and weighed using an analytical balance; the weights were recorded, and the tumors were photographed for recording. The solid tumors separated from each group of mice are shown in FIG. 19, and the corresponding tumor weight results are shown in FIG. 20. One mouse in the CDDP^{Hi} group died during the administration period, while the tumors that developed in the CDDP^{Lo}+CR108 group had the lightest weight and the minimum volume compared to the other groups; in addition, one mouse in this group exhibited complete tumor regression after the administration, indicating that CDDP^{Lo}+CR108 exhibited a significant tumor inhibitory effect, which was significantly superior to the groups where CpG and R848 were separately combined with cisplatin (the CDDP^{Lo}+C′ group and the CDDP^{Lo}+R group), and to the conventional chemotherapy with cisplatin alone (CDDP^{Hi} group), further providing relatively high safety.

### 9.3 Mouse Body Weight Measurement

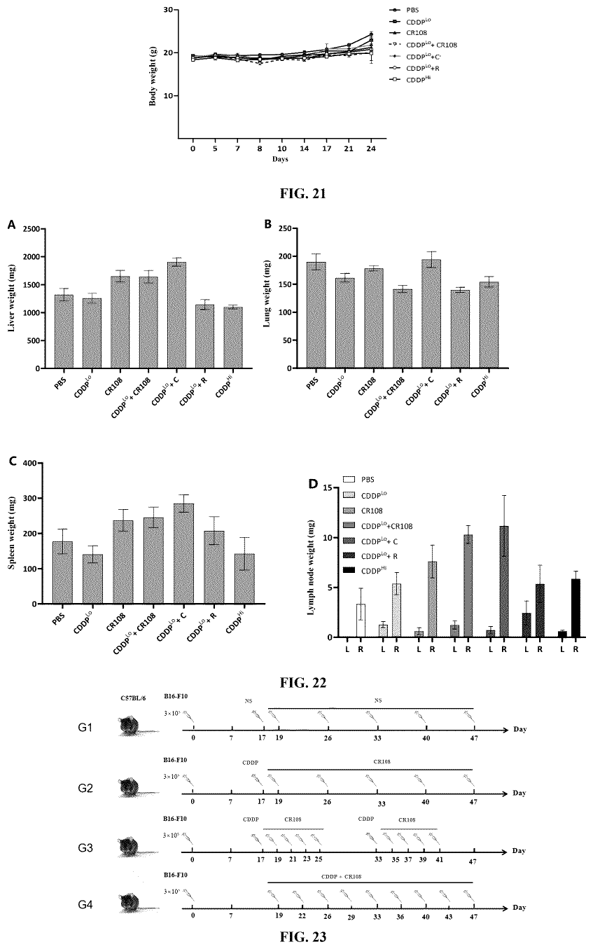

The weight results of the mice are shown in FIG. 21. There was no significant change in weight for each group, and the overall weight data remained stable with a slight increase. At the end of the experiment, there was no statistical difference in weight of the mice in each group, which indicates that the tumor adjuvant CR108 has relatively good safety.

### 9.4 Measurement of Weight of Important Mouse Organs

At the end of the experiment, the liver, lung, spleen, and lymph nodes of the mouse (the left side L indicated the lymph node at the side where the tumor was not inoculated, and the right side R indicated the lymph node at the side where the tumor was inoculated) were separated, weighed using an analytical balance, and recorded, and the weight measurement results of these mouse organs are shown in FIG. 22. As shown in FIGS. 22A-22D, the weight of the liver, lung, spleen, and lymph nodes in the CDDP^{Lo}+C group was noticeably increased to a certain extent, which was higher than that of the other groups, suggesting that CpG combined with cisplatin alone may induce an excessive immune response in the organs, leading to hypertrophy and increased weight; however, the combination of R848 and CpG combined with cisplatin (CDDP^{Lo}+CR108 group) may mitigate the excessive weight gain in the liver, lung, spleen, and lymph nodes caused by CpG combined with cisplatin alone, and simultaneously enhancing the immune response in the draining lymph nodes. This combination exhibited relatively good safety and the most effective tumor inhibitory effect.

### Example 10: Studies of Inhibitory Effect of Cisplatin Combined with CR108 Adjuvant by

Intratumoral Injection on Large-Volume Melanoma in Mice and Treatment Regimen In all the above examples, studies conducted on mouse breast cancer and melanoma models demonstrated that not only the combination of low-dose cisplatin with a tumor adjuvant (CpG+R848) provided a “toxicity reduction and efficacy enhancement” therapeutic effect on small-volume tumors (20-30 mm³), but also the tumor adjuvant therapy stimulated an internal anti-tumor immune response without the need for tumor antigen immunity. Additionally, a stable formula of the tumor adjuvant was established. However, whether a treatment regimen based on small-volume tumors is suitable for the treatment of large-volume tumors (200-300 mm³) requires further validation. Considering the clinical practice of intratumoral injection for tumor treatment, this example explored the inhibitory effect and optimal administration protocol of “low-dose chemotherapeutic drugs+tumor adjuvant” on tumors after growing to a relatively large volume (with an average of about 200 mm³) by employing various intratumoral injection protocols.

To establish a B16-F10 mouse melanoma model, the tumor cells were inoculated at 3×10⁵ cells/mouse. Once the tumors reached an appropriate size with an average of about 200 mm³, suitable mice were selected based on the tumor size and body weight and given drugs according to the following groups. The doses of the drugs were as follows: 2 μg/kg CDDP, 50 μg/mouse CpG1018, and 50 μg/mouse water-soluble R848. Specific groups were as follows: Group G1, normal saline (NS) control group: mice were treated once a week; Group G2: mice received an intratumoral injection of CDDP, followed by an intratumoral injection of CR108 two days later, once a week; Group G3: for each administration cycle, mice first received an intratumoral injection of CDDP, followed by an intratumoral injection of CR108 two days later, which was repeated every other day, with four administrations constituting one cycle, and after a one-week interruption, the next administration cycle was repeated; and Group G4: after CDDP and CR108 were mixed, mice received an intratumoral injection of the mixture, twice a week. The detailed flowchart for administration is shown in FIG. 23. After the administration was completed according to the flowchart, the administration was stopped, and the tumor size and physiological states of the mice were monitored during this period. When the tumor volume of each mouse exceeded 2000 mm³ for the first time or the body weight of the animal was less than 80% of the body weight before the first administration, it was considered an ethical death, and the animal was euthanized. The remaining mice continued to be observed.

### 10.1 Mouse Body Weight Measurement

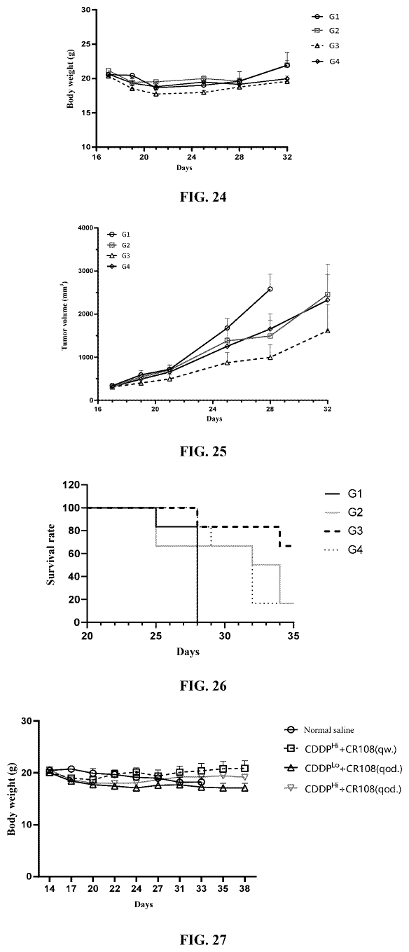

Cage-side observation and body weight measurement were continuously performed on the mice before and during the administration. It was observed in the routine cage-side observation that most of the mice exhibited transient reduced activity, piloerection, and weight loss on day 2 after the administration of the tumor adjuvant, but all returned to normal on day 3. The body weight results of the mice are shown in FIG. 24. The body weights of the mice in each group had a relatively small change range from the beginning to the end of the experiment, indicating that the low-dose cisplatin combined with CR108 has relatively good safety. In Group G1 (normal saline control group), all mice were ethically euthanized on day 28. In all groups except Group G3, more than half of the mice were ethically euthanized on day 32, marking the end of the experiment.

### 10.2 Tumor Volume Measurement

The tumor volume growth curves of the mice in each administration group are shown in FIG. 25. When the experiment reached day 28, all mice in the G1 normal saline control group died ethically because the tumor volume exceeded 2000 mm³, and the tumor volume mean was 2581.9±353.3. The tumor volume mean (Mean±SEM) of the three administration protocols and the results of T Test significance analysis relative to the G1 control group were as follows, respectively: the mean tumor volume of Group G2 was 1490±363.3, p=0.0621; the mean tumor volume of Group G3 was 996.9±286.4, p=0.0064; and the mean tumor volume of Group G4 was 1654.4±347.9, p=0.0984. The results show that when the tumors had normally reached their ethical limit, only the administration protocol used in Group G3 exhibited a significantly better tumor inhibitory effect compared to that in the normal saline control group (G1).

10.3 Survival Curves of Tumor-Bearing Mice during Treatment

As shown in FIG. 26, all mice in the normal saline control group died ethically on day 28. The median survival time for each group at the end of the experiment was as follows: 28 days for the normal saline control group, 33 days for Group G2, 32 days for Group G4, and 38 days for Group G3. As can be seen from the data that the administration protocol used in Group G3 can significantly extend the survival time of the mice bearing large-volume melanoma.

In this example, we initially identified a protocol G3 for intratumoral administration in a large-volume tumor model, and subsequently, we continued to explore the administration protocol used in Group G3 and increase the cisplatin dose to see whether it could improve the tumor inhibitory effect.

### Example 11: Study of CR108 Adjuvant Combined with Different Doses of Cisplatin for Treatment of Large-Volume Melanoma in Mice by Intratumoral Injection

The studies in Example 10 determined the administration protocol of the tumor adjuvant combined with cisplatin for the treatment of large-volume melanoma, and this example is intended to achieve a more significant tumor inhibitory effect by optimizing the cisplatin dose.

Experimental groups were as follows: (1) a normal saline control group, with the same administration protocol as that in Group G1 of Example 10; (2) CDDP^{Hi}+CR108 (at a cisplatin dose of 0.4 mg/kg), with the same administration protocol as that in Group G2 of Example 10 (qw); (3) CDDP^{Lo}+CR108 (at a cisplatin dose of 2 μg/kg), with the same administration protocol as that in Group G3 of Example 10 (qod); and (4) CDDP^{Hi}+CR108 (0.4 mg/kg cisplatin), with the same administration protocol as that in Group G3 of Example 10 (qod); the dose of CR108 used in each group was 50 μg/mouse CpG1018 and 50 μg/mouse water-soluble R848.

### 11.1 Mouse Body Weight Measurement

The body weight results of the mice are shown in FIG. 27, and cage-side observation and body weight measurement were continuously performed on the mice before and during the administration. It was observed in the routine cage-side observation that most of the mice were generally in good condition after administration, except for one mouse in the normal saline control group and one mouse in the third group (CDDP^{Lo}+CR108 (qod.)), and that the mice exhibited transient reduced activity, piloerection, and weight loss on day 2 after each administration of the tumor adjuvant, but all returned to normal on day 3. The body weight change ranges of each group of mice from the beginning to the end of the experiment were relatively small, especially in the fourth group (CDDP^{Hi}+CR108 (qod.)) where the body weight of each mouse remained stable after administration, indicating that cisplatin at an administration dose of 0.4 mg/kg combined with CR108 has relatively good safety.

### 11.2 Tumor Volume Measurement

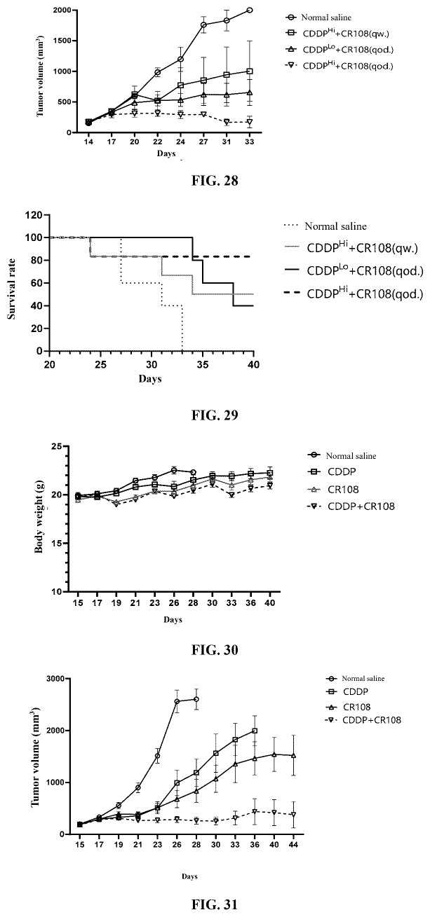

As shown in FIG. 28, when the experiment reached day 33, all mice in the normal saline control group died ethically due to tumor volume exceeding 2000 mm³, except for one mouse whose tumor was gnawed on. In group (2) (CDDP^{Hi}+CR108 (qw.)), although the cisplatin dose was increased, the tumor inhibitory effect was still inferior compared to group (3) (CDDP^{Lo}+CR108 (qod.)) and group (4) (CDDP^{Hi}+CR108 (qod.)), both of which had an increased CR108 administration frequency, further demonstrating that increasing the administration frequency of CR108 (G3) can improve the tumor inhibitory effect. Group (3) (CDDP^{Lo}+CR (qod.)), which used a low dose of cisplatin, showed a certain inhibitory effect on the tumor growth, which was substantially consistent with the administration protocol results explored in Example 10 (FIG. 25). Moreover, group (4) (CDDP^{Hi}+CR (qod.)), with an increased cisplatin dose, exhibited the most significant inhibitory effect on melanoma, with almost no tumor growth observed. It can be seen that under the same administration frequency, appropriately increasing the dose of low-dose cisplatin can significantly improve the tumor inhibitory effect.

11.3 Survival curves of tumor-bearing mice during treatment

As shown in FIG. 29, all mice in the normal saline control group died ethically on day 33. The median survival time for each group at the end of the experiment was as follows: 31 days for the normal saline control group, 37 days for the CDDP^{Hi}+CR108 (qw.) group, and 38 days for the CDDP^{Lo}+CR108 (qod.). However, the median survival time of the CDDP^{Hi}+CR108 (god.) group was still not counted on day 55 of long-term observation after two rounds of administration, indicating that this group had the best ability to prolong the survival time of tumor mice. The results show that under the same administration protocol, the survival time of mice with large-volume melanoma can be remarkably prolonged by appropriately increasing the dose of cisplatin.

### Example 12: Comparative Study of Intratumoral Injection of CR108 Adjuvant Combined with Sub-Dose of Cisplatin Versus Conventional Therapeutic Dose of Cisplatin in Treating Large-Volume Melanoma in Mice

This example mainly explored the differences between the administration protocols and optimized cisplatin doses screened in Examples 10 and 11, and the conventional therapeutic dose of cisplatin.

When the tumors grew to an appropriate size, with an average of about 200 mm³, the mice were divided into the following experimental groups for drug administration: (1) a normal saline control group, with the same administration protocol as that in Group G1 of Example 10; (2) CDDP (4 mg/kg cisplatin, conventional cisplatin chemotherapy dose), with an administration protocol being once a week; (3) CR108, with the same administration protocol as that in Group G3 of Example 10, and only saline, not cisplatin, being injected at the time point of cisplatin injection; and (4) CDDP+CR108 (0.4 mg/kg cisplatin), with the same administration protocol as that in Group G3 of Example 10; the dose of CR108 used in each group was 50 μg/mouse CpG1018 and 50 μg/mouse water-soluble R848.

### 12.1 Mouse Body Weight Measurement

Cage-side observation and body weight measurement were continuously performed on the mice before and during the administration. It was observed in the routine cage-side observation that all mice were generally in good condition after administration, and only on day 2 after each administration of the tumor adjuvant, the mice exhibited transient reduced activity, piloerection, and weight loss, but all returned to normal on day 3. Except that the body weight of the normal saline control group slightly increased due to the rapid tumor growth, the body weight of the mice in other administration groups had relatively small change ranges from the beginning to the end of the experiment, further demonstrating that the intratumoral injection of 0.4 mg/kg cisplatin combined with CR108 adjuvant has the relatively good safety. On day 28, all mice in the normal saline group had died ethically, and the rest of the mice were detected to have completed the administration. The body weight data during this period are shown in FIG. 30.

### 12.2 Tumor Volume Measurement

As shown in FIG. 31, when the experiment reached day 28, all mice in the normal saline control group died ethically due to tumor volume exceeding 2000 mm³. In group (2), where the mice received the conventional therapeutic dose of 4 mg/kg CDDP, the tumor volume was significantly lower than that in the normal saline control group on day 28, indicating a certain tumor inhibitory effect. However, on day 36, all mice in this group were euthanized due to tumor volume exceeding 2000 mm³. The tumor volume of group (3) treated with CR108 alone and group (4) treated with CDDP combined with CR108 showed significant tumor inhibitory effects by comparing the tumor volumes in the two groups with that of the normal saline control group, and the CDDP+CR108 treatment group had a significantly better tumor inhibitory effect than the CR108 single treatment group.

12.3 Survival Curves of Tumor-Bearing Mice during Treatment

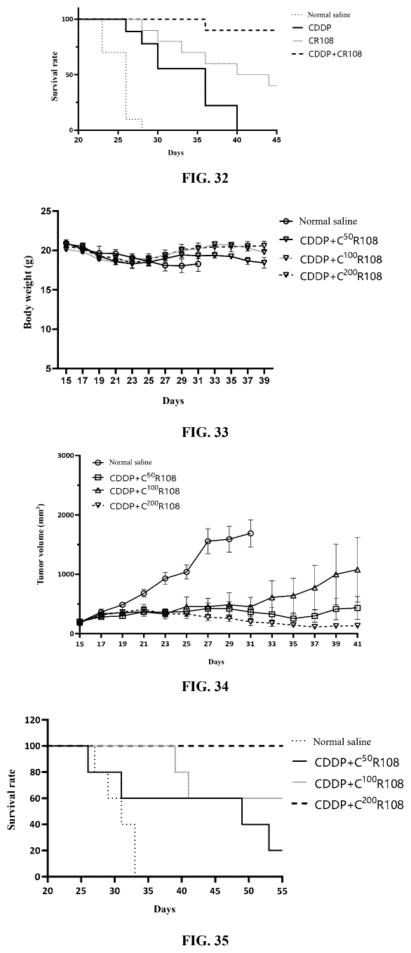

As shown in FIG. 32, all mice in the normal saline control group died ethically on day 28. The median survival time for each group at the end of the experiment was as follows: 26 days for the normal saline control group, 36 days for the CDDP group injected with the conventional therapeutic dose of 4 mg/kg cisplatin, and 42 days for the group injected with CR108 alone. However, the median survival time of the group injected with 0.4 mg/kg CDDP combined with CR108 (CDDP+CR108) was still not counted on day 45 of long-term observation after two rounds of administration, and the survival rate remained at 90%. As can be seen from the data that the CDDP+CR108 treatment group can significantly prolong the survival time of mice with large-volume melanoma compared to these two control groups, the CR108 single treatment group and the conventional cisplatin chemotherapy CDDP treatment group, and has a significant tumor inhibitory effect.

### Example 13: Study on Tumor Inhibitory Effect of Tumor Adjuvant with Different Doses of CpG1018 Combined with Fixed Dose of R848

Through a series of experiments, the cisplatin dose and the administration protocol for treating the mice with large-volume melanoma (with a tumor volume of about 200 mm³-300 mm³) by intratumoral administration were determined. Building on our previous findings, which demonstrated that CpG1018 played a major role in the overall tumor inhibitory process (as shown in FIGS. 19 and 20), this example further explored the inhibitory effects and safety of CR108 consisting of different doses of CpG1018 on the large-volume melanoma tumor model.

When the tumors grew to an appropriate size, with an average of about 200 mm³, the mice were divided into the following experimental groups for drug administration: (1) a normal saline control group, with the same administration protocol as that in Group G1 of Example 10; (2) CDDP+C⁵⁰R108 (50 μg/mouse CpG1018 and 50 μg/mouse water-soluble R848); (3) CDDP+C¹⁰⁰R108 (100 μg/mouse CpG1018 and 50 μg/mouse water-soluble R848); and (4) CDDP+C²⁰⁰R108 (200 μg/mouse CpG1018 and 50 μg/mouse water-soluble R848); the dose of CDDP used in each group was 0.4 mg/kg, and the administration protocols in groups (2), (3), and (4) were the same as that in Group G3 of Example 10.

### 13.1 Mouse Body Weight Measurement

Cage-side observation and body weight measurement were continuously performed on the mice before and during the administration. It was observed in the routine cage-side observation that all mice were generally in good condition after administration, and only on day 2 after each administration of the tumor adjuvant, the mice exhibited transient reduced activity, piloerection, and weight loss, but all returned to normal on day 3. The body weight change ranges of each group of mice from the beginning to the end of the experiment were relatively small, especially in group (4) (CDDP+C²⁰⁰R108). It indicates that the increase in the dose of CpG1018 within a certain range has very good safety. On day 33, all mice in the normal saline group had died ethically. The body weight data during this period are shown in FIG. 33.

### 13.2 Tumor Volume Measurement

The results are shown in FIG. 34. When the experiment reached day 31, the tumor volume of the mice in the normal saline control group had all exceeded 2000 mm³ or had begun to rapidly increase to the ethical death threshold, and the tumor inhibitory effects of the CR108 adjuvants consisting of different doses of CpG1018 were substantially consistent. Compared with the mice in the normal saline control group, groups (2), (3), and (4) showed significant tumor inhibitory effects on day 31, with inhibition rates of 78.51%, 73.17%, and 88.39%, respectively, on day 31. After one round of administration, there was no significant difference during the administration of different doses of CpG among the groups on day 31. After day 31, it was significant that group (4), the CDDP+C²⁰⁰R108 group, had the best anti-tumor effect, with significant inhibition of the growth and proliferation of tumor cells, and a significant reduction in tumor volume. It can be seen that the increase in the dose of CpG1018 within a certain range has a better anti-tumor effect.

13.3 Survival Curves of tumor-Bearing Mice during Treatment

As shown in FIG. 35, all mice died prior to the second round of administration in the normal saline control group; after the two administration courses were finished, the survival rates of the CpG1018 treatment groups of 50 μg, 100 μg, and 200 μg were 60%, 100%, and 100%, respectively. When the three administration courses were finished, the survival rates were 20%, 60%, and 100%, respectively. By comparing the survival status of three groups of mice, it was demonstrated that the increase in the dose of CpG1018 can significantly improve the survival time and survival rate of mice with melanoma.

In this example, with a cisplatin dose of 0.4 mg/kg and a fixed dose of R848, increasing the dose of CpG1018 to 200 μg per mouse did not result in enhanced side effects from the perspective of general mouse conditions and body weight changes, which demonstrated that the increase in the dose of CpG1018 can improve the tumor inhibitory effect and prolong the survival time of tumor-bearing mice when the same other conditions.

### Example 14: Therapeutic Effect of Cisplatin Combined with CR108 Tumor Adjuvant and Anti-PD-1 Antibody

This example further explored the effect of cisplatin combined with CR108 adjuvant and an anti-PD-1 antibody on 4T-1 breast cancer model mice. A 4T-1 mouse breast cancer model was constructed by subcutaneously inoculating tumor cells on the right side of the back of the mice proximal to the hip position at 3×10⁵ cells/mouse, and the administration was performed when the tumor volume was about 20 mm³-30 mm³ on day 5 after tumor inoculation.

Specific mouse administration groups were as follows: (1) a PBS negative control group, (2) CDDP+CR108(2 μg/kg cisplatin, 50 μg/mouse CpG1018, and 50 μg/mouse water-soluble R848), (3) CDDP+CR108+αPD-1mAb (2 μg/kg cisplatin, 50 μg/mouse CpG1018, 50 μg/mouse water-soluble R848, and 200 μg/mouse anti-PD-1 antibody), and (4) αPD-1mAb (200 μg/mouse anti-PD-1 antibody); except that the anti-PD-1 antibody was injected intraperitoneally, all other drugs were injected subcutaneously near to the tumor. In VivoMAb anti-mouse PD-1 (CD279) was from BioXcell, with catalog No. BE0273.

Administration time: The day of tumor inoculation was defined as day 0.
(1) PBS group: 100 μL of PBS was administered on day 5, day 7, day 14, and day 21.
(2) CDDP+CR108 group: 100 μL of CDDP was administered on day 5, and 100 μL of CR108 mixed drug was administered on day 7, day 14, and day 21.
(3) CDDP+CR108+αPD-1mAb group: 100 μL of CDDP was administered on day 5; 100 μL of CR108 mixed drug was administered on day 7, day 14, and day 21; and 100 μL of the anti-PD-1 antibody drug was administered on day 6, day 10, day 14, day 18, and day 22.
(4) αPD-1mAb group: 100 μL of the anti-PD-1 antibody drug was administered on day 6, day 10, day 14, day 18, and day 22.

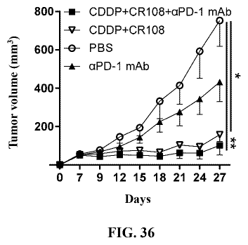

Conclusion: By measuring the tumor volume, as shown in FIG. 36, the tumor growth of the 4T-1 breast cancer model mice treated with the anti PD-1 antibody alone (αPD-1mAb group) slowed down. Compared with that, the CDDP+CR108 tumor adjuvant treatment group and the CDDP+CR108 tumor adjuvant combined with the anti PD-1 antibody treatment group (CDDP+CR108+αPD-ImAb group) significantly inhibited the growth of tumors, and there was no significant difference between the two groups on the growth of tumors in the mice.

In conclusion, the low-dose cisplatin combined with CpG and R848 treatment composition provided by the present invention not only has an excellent inhibitory effect on the growth and proliferation of triple-negative breast cancer 4T1 cells, but also can inhibit the growth and proliferation of melanoma B16 cells, suggesting that the combination therapy composition has an effect of broadly inhibiting the tumor growth. Moreover, the combination therapy composition induces the activation of B cells and T cells in tumor mice, promotes the levels of anti-tumor cytokines, IFNγ, TNFα, IFNα, perforins, and granzymes, induces a large number of CD8⁺ T cells, CD4⁺ T cells, Tfh cells, and B cells to enter tumor tissues to exert the anti-tumor effect, and is significantly superior to other groups, indicating that the combination therapy composition has an excellent effect on activating immune responses of B cells and T cells, and enables more B cells and T cells to exert effector functions, thereby inhibiting the growth of tumors. Meanwhile, the present invention provides a combination therapy method for the combination therapy composition, wherein for small-volume tumor administration, the mixed drug of CpG and R848 is administered at an interval of about 2 days after the low-dose cisplatin is used, and the mixed drug of CpG and R848 is continuously administered for a plurality of times at an interval of 7 days; for large-volume tumor administration, a certain dose of cisplatin is injected intratumorally in each administration cycle, the mixed drug of CpG and R848 is injected intratumorally at an interval of about 2 days, the administration is repeated one time every other day, with four administrations constituting one cycle, and after a one-week interruption, the next administration cycle is repeated; compared with other administration methods, the combination therapy method has the most significant effect of inhibiting the tumor growth, indicating that the combination therapy method provided by the present invention has a better effect of inhibiting the tumor growth. Moreover, the combination therapy composition and the combination therapy method therefor provided by the present invention can not only significantly inhibit the growth of proximal tumors, but also significantly inhibit the growth of distal tumors, indicating that the combination therapy composition and the combination therapy method therefor have inhibitory effects on systemic tumor lesions, and further demonstrating that the combination therapy composition effectively activates systemic anti-tumor immune responses and reactions, which is not only effective on local tumor lesions, but also has efficient anti-tumor immune effects on tumor lesions that spread throughout the body. The above effects are applicable to research-grade R848 (water soluble) and pharmaceutical grade R848 (lipid soluble), as well as research-grade CpG1826 and pharmaceutical grade CpG1018.

Accordingly, the present invention provides a combination therapy composition and a combination therapy method for treating a tumor, wherein the combination therapy composition is a low-dose chemotherapeutic drug, cisplatin, combined with CpG and R848; the combination therapy method involves the use of cisplatin, CpG, and R848 at specific times, that is, the low-dose chemotherapeutic drug, cisplatin, is first used to kill a tumor so as to release a tumor neoantigen, enabling the immune system of an organism to autonomously identify and screen the neoantigen, and then CpG and R848 immune activators are used to induce and promote the organism to generate an immune response specific to the tumor neoantigen, such that the anti-tumor function of T cells is improved and strengthened, thereby achieving the effect of inhibiting the tumor growth.

Finally, it should be noted that, the above examples are only used to illustrate the embodiments of the present disclosure, and should not limit the same. Although the present disclosure is described in detail with reference to the examples described above, it will be appreciated by those skilled in the art that, the embodiments in the examples described above can still be modified, or some or all of the technical features can be equivalently substituted. Such modifications or substitutions do not make the embodiments corresponding thereto depart from the scope of the embodiments in the examples of the present invention.

1. A pharmaceutical composition, comprising a therapeutically effective amount of a platinum-based chemotherapeutic drug, a therapeutically effective amount of a CpG oligonucleotide, and a therapeutically effective amount of R848.

2. The pharmaceutical composition according to claim 1, wherein the platinum-based chemotherapeutic drug is selected from one or more of cisplatin, carboplatin, nedaplatin, oxaliplatin, and lobaplatin; preferably, the platinum-based chemotherapeutic drug is cisplatin.

3. The pharmaceutical composition according to claim 1, wherein the therapeutically effective amount of the CpG oligonucleotide (CpG ODN) is a B-class CpG ODN or a C-class CpG ODN;
preferably, the B-class CpG ODN is selected from one or more of ODN 1826, ODN 1018, and ODN 2006/7909;
preferably, the C-class CpG ODN is selected from one or more of ODN M362, ODN 2395, and D-SL03.

4. The pharmaceutical composition according to claim 1, wherein the therapeutically effective amount of R848 is selected from one or more of water-soluble R848 and lipid-soluble R848.

5. The pharmaceutical composition according to claim 1, wherein the therapeutically effective amount of the platinum-based chemotherapeutic drug is administered at a dose of 2 μg/kg to 4000 μg/kg;
the therapeutically effective amount of the CpG oligonucleotide is administered at a dose of 0.001 mg/time to 32 mg/time;
the therapeutically effective amount of R848 is administered at a dose of 0.001 mg/time to 5 mg/time;
preferably, the therapeutically effective amount of the platinum-based chemotherapeutic drug is administered at a dose of 2 μg/kg to 400 μg/kg;
the therapeutically effective amount of the CpG oligonucleotide is administered at a dose of 0.05 mg/time to 2 mg/time;
the therapeutically effective amount of R848 is administered at a dose of 0.01 mg/time to 1 mg/time.

6. The pharmaceutical composition according to claim 1, wherein the therapeutically effective amount of the platinum-based chemotherapeutic drug, the therapeutically effective amount of the CpG oligonucleotide, and the therapeutically effective amount of R848 are sequentially administered to a subject in need thereof;
alternatively, the administrations of the therapeutically effective amount of the CpG oligonucleotide and the therapeutically effective amount of R848 are performed simultaneously, and the administration of the therapeutically effective amount of the platinum-based chemotherapeutic drug is performed prior to the administrations of the therapeutically effective amount of the CpG oligonucleotide and the therapeutically effective amount of R848.

7. The pharmaceutical composition according to claim 6, wherein the therapeutically effective amount of the platinum-based chemotherapeutic drug is administered 1 time, and the therapeutically effective amount of the CpG oligonucleotide and the therapeutically effective amount of R848 are administered 1-5 times for 1 administration cycle;
a time interval between the first administration time of the therapeutically effective amount of the CpG oligonucleotide and the therapeutically effective amount of R848 and the first administration time of the therapeutically effective amount of the platinum-based chemotherapeutic drug is 1-5 days;
preferably, a time interval between the first administration time of the therapeutically effective amount of the CpG oligonucleotide and the therapeutically effective amount of R848 and the first administration time of the therapeutically effective amount of the platinum-based chemotherapeutic drug is 2 days;
a time interval for each administration of the first to fifth administrations of the therapeutically effective amount of the CpG oligonucleotide and the therapeutically effective amount of R848 is 1-14 days;
the administration cycle for the subject is 1-3 times, and an interval for each administration cycle is 5-7 days.

8. The pharmaceutical composition according to claim 1, wherein a mode of administration of the pharmaceutical composition comprises one or more of subcutaneous administration near a tumor and intratumoral injection administration.

9. The pharmaceutical composition according to claim 1, further comprising an additional tumor therapeutic drug, wherein preferably, the additional tumor therapeutic drug is an immunotherapeutic drug, and further preferably, the immunotherapeutic drug is a PD-1/PD-L1 inhibitor.

10. A kit for treating a tumor or cancer, comprising a therapeutically effective amount of a platinum-based chemotherapeutic drug, a therapeutically effective amount of a CpG oligonucleotide, and a therapeutically effective amount of R848, wherein
preferably, the platinum-based chemotherapeutic drug is selected from one or more of cisplatin, carboplatin, nedaplatin, oxaliplatin, and lobaplatin; preferably, the platinum-based chemotherapeutic drug is cisplatin; the therapeutically effective amount of the CpG oligonucleotide (CpG ODN) is a B-class CpG ODN or a C-class CpG ODN;
more preferably, the B-class CpG ODN is selected from one or more of ODN 1826, ODN 1018, and ODN 2006/7909;
more preferably, the C-class CpG ODN is selected from one or more of ODN M362, ODN 2395, and D-SL03;
the therapeutically effective amount of R848 is selected from one or more of water-soluble R848 and lipid-soluble R848.

11. The kit for treating the tumor or cancer according to claim 10, wherein the therapeutically effective amount of the platinum-based chemotherapeutic drug, the therapeutically effective amount of the CpG oligonucleotide, and the therapeutically effective amount of R848 are packaged separately, or the therapeutically effective amount of the platinum-based chemotherapeutic drug is packaged individually, and the therapeutically effective amount of the CpG oligonucleotide and the therapeutically effective amount of R848 are packaged in a mixed manner;
preferably, the therapeutically effective amount of the platinum-based chemotherapeutic drug, the therapeutically effective amount of the CpG oligonucleotide, and the therapeutically effective amount of R848 are sequentially administered to a subject in need thereof;
more preferably, the administrations of the therapeutically effective amount of the CpG oligonucleotide and the therapeutically effective amount of R848 are performed simultaneously, and the administration of the therapeutically effective amount of the platinum-based chemotherapeutic drug is performed prior to the administrations of the therapeutically effective amount of the CpG oligonucleotide and the therapeutically effective amount of R848;
more preferably, the therapeutically effective amount of the platinum-based chemotherapeutic drug is administered 1 time, and the therapeutically effective amount of the CpG oligonucleotide and the therapeutically effective amount of R848 are administered 1-5 times for 1 administration cycle;
a time interval between the first administration time of the therapeutically effective amount of the CpG oligonucleotide and the therapeutically effective amount of R848 and the first administration time of the therapeutically effective amount of the platinum-based chemotherapeutic drug is 1-5 days;
preferably, a time interval between the first administration time of the therapeutically effective amount of the CpG oligonucleotide and the therapeutically effective amount of R848 and the first administration time of the therapeutically effective amount of the platinum-based chemotherapeutic drug is 2 days;
a time interval for each administration of the first to fifth administrations of the therapeutically effective amount of the CpG oligonucleotide and the therapeutically effective amount of R848 is 1-14 days;
the administration cycle for administering the therapeutically effective amount of the CpG oligonucleotide and the therapeutically effective amount of R848 to the subject is 1-3 times, and an interval for each administration cycle is 5-7 days.

12. The kit for treating the tumor or cancer according to claim 10, further comprising an additional tumor therapeutic drug, wherein preferably, the additional tumor therapeutic drug is an immunotherapeutic drug, and further preferably, the immunotherapeutic drug is a PD-1/PD-L1 inhibitor.

13. (canceled)

14. (canceled)

15. A method for treating a tumor or cancer, comprising administering to a subject a therapeutically effective amount of a platinum-based chemotherapeutic drug, a therapeutically effective amount of a CpG oligonucleotide, and a therapeutically effective amount of R848, wherein
preferably, the platinum-based chemotherapeutic drug is selected from one or more of cisplatin, carboplatin, nedaplatin, oxaliplatin, and lobaplatin; preferably, the platinum-based chemotherapeutic drug is cisplatin; the therapeutically effective amount of the CpG oligonucleotide (CpG ODN) is a B-class CpG ODN or a C-class CpG ODN;
more preferably, the B-class CpG ODN is selected from one or more of ODN 1826, ODN 1018, and ODN 2006/7909;
more preferably, the C-class CpG ODN is selected from one or more of ODN M362, ODN 2395, and D-SL03;
the therapeutically effective amount of R848 is selected from one or more of water-soluble R848 and lipid-soluble R848.

16. The method for treating the tumor or cancer according to claim 15, wherein the therapeutically effective amount of the platinum-based chemotherapeutic drug is administered at a dose of 2 μg/kg to 4000 μg/kg;
the therapeutically effective amount of the CpG oligonucleotide is administered at a dose of 0.001 mg/time to 32 mg/time;
the therapeutically effective amount of R848 is administered at a dose of 0.001 mg/time to 5 mg/time;
preferably, the therapeutically effective amount of the platinum-based chemotherapeutic drug is administered at a dose of 2 μg/kg to 400 μg/kg;
the therapeutically effective amount of the CpG oligonucleotide is administered at a dose of 0.05 mg/time to 2 mg/time;
the therapeutically effective amount of R848 is administered at a dose of 0.01 mg/time to 1 mg/time.

17. The method for treating the tumor or cancer according to claim 16, wherein the therapeutically effective amount of the platinum-based chemotherapeutic drug, the therapeutically effective amount of the CpG oligonucleotide, and the therapeutically effective amount of R848 are packaged separately, or the therapeutically effective amount of the platinum-based chemotherapeutic drug is packaged individually, and the therapeutically effective amount of the CpG oligonucleotide and the therapeutically effective amount of R848 are packaged in a mixed manner;
preferably, the therapeutically effective amount of the platinum-based chemotherapeutic drug, the therapeutically effective amount of the CpG oligonucleotide, and the therapeutically effective amount of R848 are sequentially administered to a subject in need thereof;
more preferably, the administrations of the therapeutically effective amount of the CpG oligonucleotide and the therapeutically effective amount of R848 are performed simultaneously, and the administration of the therapeutically effective amount of the platinum-based chemotherapeutic drug is performed prior to the administrations of the therapeutically effective amount of the CpG oligonucleotide and the therapeutically effective amount of R848;
more preferably, the therapeutically effective amount of the platinum-based chemotherapeutic drug is administered 1 time, and the therapeutically effective amount of the CpG oligonucleotide and the therapeutically effective amount of R848 are administered 1-5 times for 1 administration cycle;
a time interval between the first administration time of the therapeutically effective amount of the CpG oligonucleotide and the therapeutically effective amount of R848 and the first administration time of the therapeutically effective amount of the platinum-based chemotherapeutic drug is 1-5 days;
preferably, a time interval between the first administration time of the therapeutically effective amount of the CpG oligonucleotide and the therapeutically effective amount of R848 and the first administration time of the therapeutically effective amount of the platinum-based chemotherapeutic drug is 2 days;
a time interval for each administration of the first to fifth administrations of the therapeutically effective amount of the CpG oligonucleotide and the therapeutically effective amount of R848 is 1-14 days;
the administration cycle for administering the therapeutically effective amount of the CpG oligonucleotide and the therapeutically effective amount of R848 to the subject is 1-3 times, and an interval for each administration cycle is 5-7 days.

18. The method for treating the tumor or cancer according to claim 16, wherein the tumor or cancer is selected from breast cancer, melanoma, liver cancer, basal cell carcinoma, cutaneous squamous cell carcinoma, cutaneous T-cell lymphoma, and colorectal cancer; preferably, the tumor or cancer is selected from breast cancer and melanoma.
